(19) United States
(12) Patent Application Publication
(10) Pub. No.: US 2025/0372201 A1
(43) Pub. Date: Dec. 4, 2025

(54) METHODS AND SYSTEMS FOR ANALYSIS OF RECEPTOR INTERACTION

(71) Applicant: Regeneron Pharmaceuticals, Inc., Tarrytown, NY (US)

(72) Inventor: Peter Hawkins, Tarrytown, NY (US)

(21) Appl. No.: 19/225,704

(22) Filed: Jun. 2, 2025

Related U.S. Application Data
(60) Provisional application No. 63/654,241, filed on May 31, 2024.

(51) Int. Cl. G16B 20/30; G16B 15/30; G16B 40/20

(52) U.S. Cl. CPC G16B 20/30; G16B 15/30; G16B 40/20

(57) ABSTRACT

A computational framework for high-throughput mapping, validating, and predicting receptor sequence interactions is described. A method includes pre-processing sequence data, adjusting data for noise, generating intermediate strength of interaction data, aggregating the intermediate strength of interaction data based on dextramer clustering and based on TCR clustering, and generating final relative strength of interaction data that identifies reliable TCR-pMHC binding events.

### CROSS-REFERENCE TO RELATED APPLICATION

This application claims the benefit of priority of U.S. Provisional Application No. 63/654,241 filed May 31, 2024, the content of which is incorporated in its entirety herein.

### BACKGROUND

T cell antigen specificity, mediated via T cell receptors (TCRs), is a hallmark of cellular immunity. TCRs are heterodimeric proteins found on the T cell surface, commonly comprised of an α- and β-chain. The TCR α- and β-chain genes are composed of discrete V, D (β-chain only) and J segments that are joined by somatic recombination during T cell development. This genetic rearrangement generates a highly diverse TCR repertoire (estimated to range from 1015 to 1061 possible receptors in human) to ensure efficient control of viral infections and other pathogen-induced diseases. TCR diversity is primarily exhibited in complementarity determining region (CDR) loops (CDR1, CDR2 and CDR3) on two chains of the TCR, which may be alpha and beta chains, or gamma and delta chains (encoded by TR(A/B/G/D)). TCRs engage peptides that are presented by major histocompatibility complex (MHC) proteins, and therefore directly determines the specificity of T cell pMHC binding.

Although the factors underlying TCR-pMHC recognition are not fully understood, recent studies have shown that T cells binding to a particular pMHC share common TCR sequence features and, in select cases, it is possible to predict the specific binding probability of an unseen TCR sequence based on learned TCR sequence features. However, these studies were limited by the quantity and diversity of training data generated by traditional single multimer sorting or antigen re-exposure assays. Further understanding of TCR-pMHC specific binding requires innovation in both computational and experimental methods. 10x Genomics recently published a dataset generated from their highly multiplexed pooled dextramer binding immune profiling platform that couples feature-barcoded dextramers and single cell TCR sequencing. This approach makes it feasible to generate high-dimensional pMHC specific binding data at the single cell level with paired T cell α- and β-chain sequences, whereas other large-scale pooled multimer approaches only estimate the composition of pMHC specific binding T cells.

As with any other high throughput technology, highly multiplexed dextramer binding data are often associated with low signal-to-noise ratios. This makes it bioinformatically challenging to reliably identify TCR-pMHC binding events using such large-scale binding datasets. Unexpectedly high cross-HLA and cross-pMHC associations were observed from the binding events that 10x Genomics provided. This low signal-to-noise dataset calls for more sophisticated computational normalization methods to discriminate true TCR-pMHC binding events from non-specific background.

As next-generation screening technologies have increased the volume of available TCR-pMHC binding data, state-of-the-art functional classifiers to computationally validate and subsequently predict TCR-pMHC specific recognition have become more feasible. While the results from initial TCR-pMHC binding classifiers are encouraging, they were only trained using CDR loop sequences and thus unable to learn the overall complex sequence patterns from full-length TCR sequences, resulting in sub-optimal prediction accuracy for highly diverse pMHC binding TCRs. Leveraging the ability of deep learning methods to learn complex patterns, several deep learning frameworks were recently proposed to uncover binding patterns in large, highly complex TCR sequence datasets.

In this study, a computational framework for mapping, computationally validating, and predicting TCR-pMHC specific recognition using highly multiplexed dextramer binding data is described.

### BRIEF SUMMARY

Disclosed are methods comprising pre-processing sequence data, adjusting data for noise, generating intermediate strength of interaction data, aggregating the intermediate strength of interaction data based on dextramer clustering and based on TCR clustering, and generating final relative strength of interaction data that identifies reliable TCR-pMHC binding events.

Disclosed are methods comprising performing droplet-based single-cell RNA sequencing to generate, for each droplet, RNA sequence data, TCR sequence data, and dextramer sequence data; determining, based on a first measure of similarity, one or more TCR clusters from the TCR sequence data; determining, based on a second measure of similarity, one or more dextramer clusters from the dextramer sequence data; creating RNA data indicating a count of RNA sequences derived from one or more genes present in each cell containing droplet; creating dextramer data indicating a count of each of one or more dextramers present in each cell containing droplet; creating TCR data indicating one or more TCR sequences present in each cell containing droplet; adjusting, based on background correction derived from counts of dextramers present in non-cell containing droplets, the dextramer data; removing, based on the RNA data, data associated with a droplet comprising two or more cells and data associated with a droplet containing no TCR sequence from the dextramer data; normalizing the dextramer data, generating, based on the dextramer data, intermediate relative strength of interaction data indicating a strength of interaction for a TCR with each of one or more dextramers; removing, from the intermediate relative strength of interaction data, data that does not satisfy a threshold; aggregating, based on the one or more dextramer clusters, data of the intermediate relative strength of interaction data; removing, from the intermediate relative strength of interaction data, data that does not satisfy an interaction threshold; creating final strength of interaction data comprising data remaining in the intermediate relative strength of interaction data that indicates a strength of interaction for a TCR with each of one or more dextramer clusters; aggregating, based on the one or more TCR clusters, data of the intermediate relative strength of interaction data; removing, from the intermediate relative strength of interaction data, data that does not satisfy a clonal specificity threshold; adding, to the final strength of interaction data, data remaining in the intermediate relative strength of interaction data that indicates a strength of interaction for a TCR cluster with each of one or more dextramer clusters; and outputting the final strength of interaction data.

Disclosed are methods comprising performing TCR-pMHC binding specificity data normalization on dextramer sequence data to identify a plurality of TCR-pMHC binding events; determining, based on the dextramer sequence data, a training dataset comprising a plurality of TCR sequences wherein each TCR sequence is associated with a binding affinity; determining, based on the plurality of TCR sequences, a plurality of features for a predictive model; training, based on a first portion of the training dataset, the predictive model according to the plurality of features; testing, based on a second portion of the training dataset, the predictive model; and outputting, based on the testing, the predictive model.

Disclosed are methods comprising presenting, to a trained predictive model, an unknown TCR sequence, wherein the trained predictive model is trained based on a training data set derived according to the disclosed methods; and predicting, by the trained predictive model, a binding affinity.

Disclosed are apparatuses configured to perform any of the disclosed methods.

Disclosed are computer readable media having processor-executable instructions embodiment thereon configured to cause an apparatus to perform any of the disclosed methods.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example operational environment.

FIG. 2A shows an experimental approach for generating multi-omics, high-throughput TCR-pMHC binding data.

FIG. 2B shows an example droplet-based single cell RNA sequencing process.

FIG. 3 shows example droplets that contain T cells.

FIG. 4 shows an example method.

FIG. 5A shows example RNA data.

FIGS. 5B shows example TCR data.

FIG. 5C shows example dextramer data.

FIG. 6 shows an example 2-component mixture model.

FIG. 7 shows an example of background subtraction and normalization.

FIG. 8A shows an example of intermediate strength of interaction data.

FIG. 8B shows a result of aggregating, based on the one or more dextramer clusters, data of the intermediate relative strength of interaction data.

FIG. 8C shows a result of aggregating, based on the one or more TCR clusters, data of the intermediate relative strength of interaction data.

FIG. 9 shows an example of final strength of interaction data.

FIG. 10 shows an example machine learning method.

FIG. 11 shows an example machine learning method.

FIG. 12A shows an application of the disclosed methods in a low noise scenario.

FIG. 12B shows an application of the disclosed methods in a medium noise scenario.

FIG. 12C shows an application of the disclosed methods in a high noise scenario.

FIG. 13 shows an example operating environment.

FIG. 14 shows an example method.

### DETAILED DESCRIPTION

The disclosed method and compositions may be understood more readily by

reference to the following detailed description of particular embodiments and the Example included therein and to the Figures and their previous and following description.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a TCR” includes a plurality of such TCRs, reference to “the dextramer” is a reference to one or more dextramers and equivalents thereof known to those skilled in the art, and so forth.

The term “subject” or “donor” may refer to an animal, such as a mammalian species (preferably human) or avian (e.g., bird) species. More specifically, a subject or donor can be a vertebrate, e.g., a mammal such as a mouse, a primate, a simian or a human. Animals include farm animals, sport animals, and pets. A subject or donor can be a healthy individual, an individual that has symptoms or signs or is suspected of having a disease or a predisposition to the disease, or an individual that is in need of therapy or suspected of needing therapy. In some embodiments, the subject donor is human, such as a human who has, or is suspected of having, cancer.

The term “Unique Molecular Identifier (UMI)” or “barcode” as used herein, generally refers to a label that may be attached to a molecule (e.g., dextramer, cell) to convey information about the molecule. For example, a UMI can be a polynucleotide sequence attached to each dextramer and a common sequencing UMI can be a polynucleotide sequence attached during sequencing. This UMI can then be sequenced. The presence of the same UMI on multiple sequences may provide information about the origin of the sequence. For example, a UMI may indicate that the sequence came from a particular dextramer. A UMI can also indicate that a sequence came from a particular cell/dextramer combination.

As used herein, the terms “sequencing” or “sequencer” refer to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina or Applied Biosystems.

A “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

The term “DNA (deoxyribonucleic acid)” refers to a chain of nucleotides comprising deoxyribonucleosides that each comprise one of four nucleobases, namely, adenine (A), thymine (T), cytosine (C), and guanine (G). The term “RNA (ribonucleic acid)” refers to a chain of nucleotides comprising four types of ribonucleosides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “nucleotide sequence”, “genomic sequence,” “genetic sequence,” or “fragment sequence,” or “nucleic acid sequencing read” denotes any information or data that is indicative of the order of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

“Optional” or “optionally” means that the subsequently described event, circumstance, or material may or may not occur or be present, and that the description includes instances where the event, circumstance, or material occurs or is present and instances where it does not occur or is not present.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Exemplary” means “an example of” and is not intended to convey an indication of a preferred or ideal configuration. “Such as” is not used in a restrictive sense, but for explanatory purposes.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

### B. Methods of Identifying Reliable Receptor-pMHC Binding and Uses Thereof

In some aspects, the methods and systems described can identify reliable TCR-pMHC bindings by analyzing multi-omics high-throughput binding data. The methods and systems may be referred to herein as ICONv2 (Integrative COntext-specific Normalization).

Assigning reactivity of T cell receptors (TCRs) to antigens from multiplexed high throughput screening requires statistical analysis of raw data and accurate consideration of underlying biologic interactions. Provided herein are improvements to dextramer technology; furthermore, the disclosed methods find equal application to the interaction of T cell receptors or B cell receptors to antigens. The methods disclosed herein generally relate to counting numbers of dextramer (molecules containing multiple peptide: MHC protein complexes) associated with a single T cell. Demonstrated technological improvements of the present methods relate at least to: (1) the removal of background noise per dextramer in a screen and normalization of signals across dextramers, and/or (2) recognizing that a TCR that is able to bind several dextramers that have similar peptide sequences should not be penalized for having non-specific binding, since TCR cross-reactivity to such peptides should be expected from a biological perspective. Such improvements may be particularly important in cancer applications where many similar neo-epitopes may be included in a panel for high-throughput screening. To this end, the disclosed methods accurately assign TCR/BCRs to their antigen reactivity based on high-throughput experimental TCR/BCR to antigen reactivity screens.

### 1. Data Acquisition

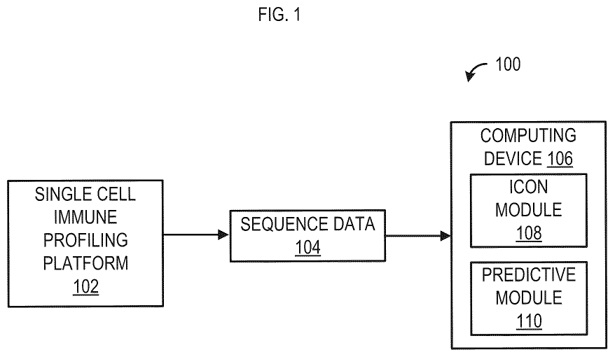

Disclosed are methods of acquiring, receiving, and/or determining multi-omics high-throughput binding data. As shown in FIG. 1, a system 100 can comprise a single-cell immune profiling platform 102. The single-cell immune profiling platform 102 may be configured to generate multi-omics high-throughput binding data (e.g., sequence data 104). In an aspect, the multi-omics high-throughput binding data can comprise one or more of single cell sequence data, dextramer sequence data, and/or single cell receptor sequence data. The single cell sequence data can comprise, for example, RNA-seq data. The dextramer sequence data can comprise, for example, dCODE-Dextramer-seq and/or cell surface protein expression sequencing, also referred to as CITE-seq (cellular indexing of transcriptomes and epitopes by sequencing). The single cell receptor sequence data can comprise, for example, TCR-seq data, such as paired αβ chain (or γδ chain) single cell TCR-seq data.

In some aspects, the multi-omics high-throughput binding data can be previously generated and incorporated into the disclosed methods. In some aspects, the multi-omics high-throughput binding data can be generated as part of the disclosed methods.

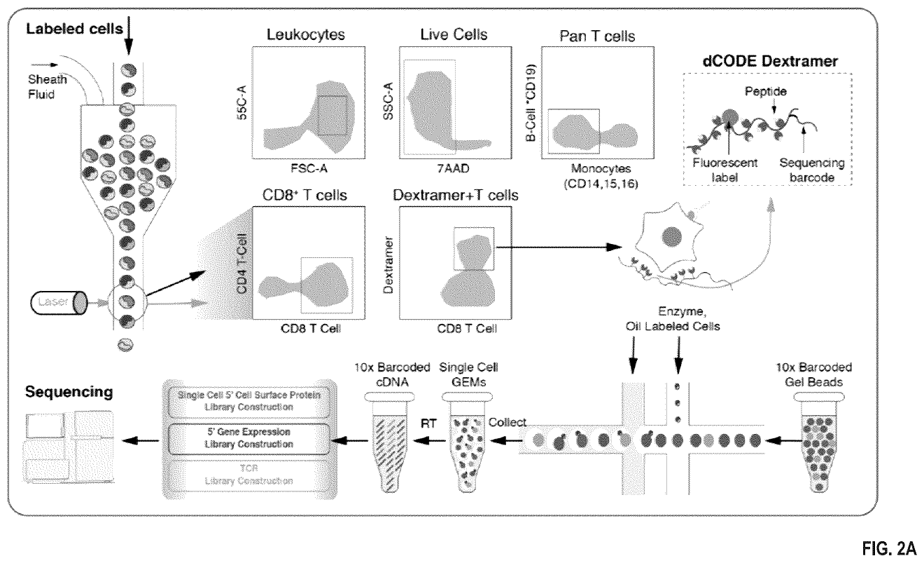

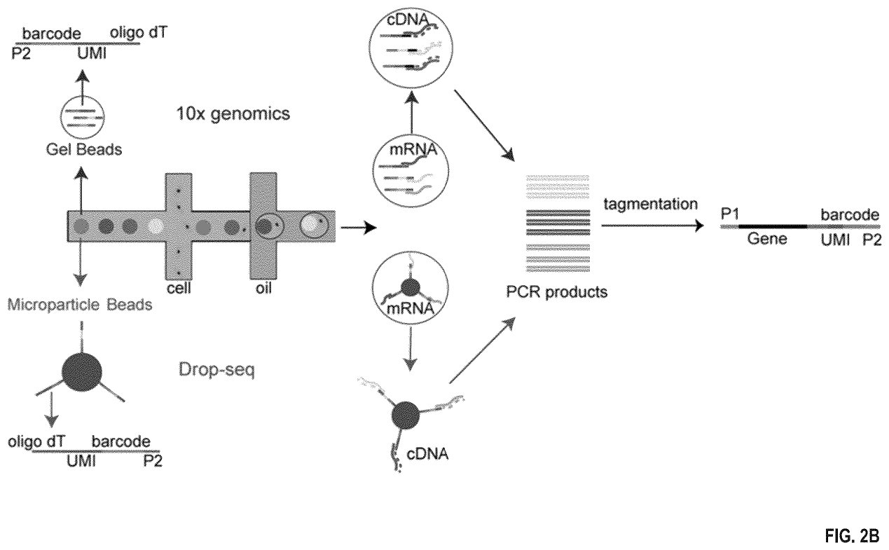

In some aspects, as shown in FIG. 2A, the single-cell immune profiling platform 102 may be configured to label peripheral blood mononuclear cells (PBMCs) from healthy human donors for sorting on cells, such as, T cells or B cells. In some aspects, the cells can be T cells (e.g., CD4+ or CD8+ cells). In some aspects, the T cells can be αβ T cells or γδ T cells. In some aspects, the cells can be B cells. Thus, when labeling for sorting, the label can be a CD4, CD8, or B cell specific label.

PBMC T cells from healthy human donors were labeled for sorting on CD8+ cells. Sorted CD8+ T cells were stained with a pool of 50 dCODE Dextramer antibodies. Dextramer positive CD8+ T cells were sorted by flow cytometry and were captured individually as input for the 10x Genomics single cell sequencing library preparation. Three libraries were generated for gene expression, cell surface protein/dCODE expression, paired TCR sequences for each CD8+ T cell.

In some aspects, once the cell type of interest has been sorted, the sorted cells can then be sorted for cells that bind a particular peptide-major histocompatibility complex (MHC) (pMHC). In some aspects, cells can be combined with a set of dextramers, for example, dCODE™ dextramers. In some aspects, the dCODE™ Dextramer® technology can be used. The dextramers can comprise two or more MHCs, a peptide presented by each MHC, and a DNA barcode. In some aspects, a pool of dextramers are used. In some aspect, a pool of dextramers can comprise, but is not limited to, 2, 3, 4, 5, 6,7, 8, 9, 10, 15, 20, 25, 30, 35, 40, 45, 50, 55, 60, 65, 70,75, 80, 85, 90, 95, or 100 single dextramers each comprising a different pMHC. In some aspects, a pool of dextramers comprises two or more of each of the single dextramers comprising a different pMHC. In some aspects, the two or more MHCs on a single dextramer are the same and therefore present the same peptide. In some aspects the MHC can be a MHC class I (MHC I) or MHC class II (MHC II). In some aspects, the DNA barcode comprises one or more primer sequences, a peptide-MHC (pMHC) specific barcode, and a unique molecular identifier. In some aspects, the dextramers can further comprise a label. For example, the label can be a fluorescent label. In some aspects, cells that bind a particular pMHC are sorted based on the label on the dextramer. In some aspects, cells that bind a particular pMHC are sorted based on a labeled antibody specific to the dextramer.

In some aspects, the cell sorting for specific cell types and the cell sorting for cells recognizing a dextramer can be performed simultaneously or consecutively.

In some aspects, after sorting of the cells that bound to dextramers comprising pMHCs, each cell and the corresponding dextramer can be sequenced. In some aspects, the cell sequence and the dextramer sequence (e.g., the DNA barcode sequence from the dextramer) all have a common sequencing barcode which allows one to determine which cell sequences were associated with which dextramer sequences. In some aspects, the Next GEM technology can be used for sequencing. The common sequencing barcode is different than the DNA barcode found on the dextramers.

In some aspects, the sequencing of the cells that bound to dextramers comprising pMHCs provides the sequence data 104 which may comprise single cell sequence data, dextramer sequence data, single cell receptor sequence data, combinations thereof, and the like. In some aspects, the single cell sequence data comprises sequences from the entire cellular genome or transcriptome. Thus, in some aspects, single cell sequence data comprises gene expression data. In some aspect, single cell sequence data comprise RNA sequence data. In some aspects, the dextramer sequence data comprises a dextramer sequence, a DNA barcode sequence, and/or the like. In some aspects, single cell receptor sequence data comprises a sequence of a specific receptor. For example, single cell receptor sequence data comprises single cell TCR or B cell receptor (BCR) sequence data. In some aspects, single cell TCR sequence data comprises paired TCR sequence data. In some aspects, paired TCR sequence data comprises sequence data for the a chain and the β chain, if present, for each cell. In some aspects, paired TCR sequence data comprises sequence data for the γ chain and the δ chain, if present, for each cell. Thus, for each method and example described herein, the sequencing of the alpha chains and beta chains can be exchanged for sequencing of the gamma chains and delta chains.

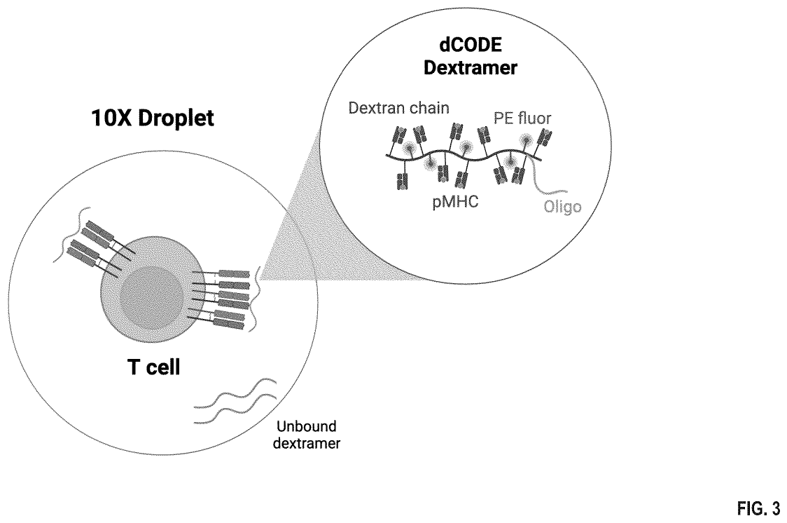

In some aspects, as shown in FIG. 2B, the single-cell immune profiling platform 102 may be configured for droplet-based single cell RNA sequencing. Microfluidics is a newly developed, highly integrated system that allows sequential processing of small volumes of fluids in channels with dimensions of tens to hundreds of micrometers to achieve single cell culture and sequencing. Several microfluidics platforms are available, such as the Fluidigm C1, Drop-seq, and 10x Genomics Chromium. As shown in FIG. 2B, in the Drop-seq, one channel contains single cells for analysis and the other contains microparticle beads. The surface of a microparticle bead binds oligonucleotides that consist of oligo dT (green), a unique molecular identifier (UMI; red), a cell barcode (blue), and a PCR primer (brown). Immediately after droplet formation, cells are lysed and mRNAs released and then hybridized with oligonucleotides on the surface of the microparticle beads based on oligo dT binding. Droplets are then broken and mRNAs are reverse-transcribed in bulk and amplified for sequencing using PCR. Moreover, in the 10x Genomics platform, one channel contains single cells for analysis and the other contains gel beads mixed with oligonucleotides that consist of oligo dT, UMI, cell barcode, and a PCR primer. Cells and reagents are next mixed with gel beads. After cell lysis, their mRNAs are released and hybridized with oligonucleotides based on oligo dT binding, and are next reverse-transcribed in bulk and amplified for sequencing using PCR. P1 and P2 are PCR primers for establishing libraries for sequencing. As shown in FIG. 3, droplets contain T cells and potentially multiple pMHC on dextran chains. A single droplet may contain one or more dextramers bound to the cell surface from one or more pMHC (color corresponds to pMHC), one oligo label per pMHC, and “background” dextramers that may not bind the T cell, i.e. by binding to its TCR. The methods and systems disclosed are essential to analyze such data and understand true TCR-pMHC binding events.

Returning to the system 100 shown in FIG. 1, in an aspect, the sequence data 104 may be provided to a computing device 106. The computing device 106 may be, for example, a smartphone, a tablet, a laptop computer, a desktop computer, a server computer, or the like. The computing device 106 may include a group of one or more servers. The computing device 106 may be configured to generate, store, maintain, and/or update various data structures including a database for storage of one or more of the sequence data 102. The computing device 106 may be configured to operate one or more application programs, such as an Integrative COntext-specific Normalization (ICONv2) module 108 and/or a predictive module 110. The ICONv2 module 108 and the predictive module 110 may be stored and or configured to operate on the same computing device or separately on separate computing devices.

In some aspects, the ICONv2 module 108 can be configured to analyze the received sequence data 104 (e.g., multi-omics high-throughput binding data, RNA sequence data, dextramer sequence data, TCR sequence data, etc.). The sequence data 104 may include sequence information as well as meta information. The sequence data 104 can be stored in any suitable file format including, for example, VCF files, FASTA files or FASTQ files, as are known to those of skill in the art. FASTA and FASTQ are common file formats used to store raw sequence reads from high throughput sequencing. FASTQ files store an identifier for each sequence read, the sequence, and the quality score string of each read. FASTA files store the identifier and sequence only. Other file formats are contemplated.

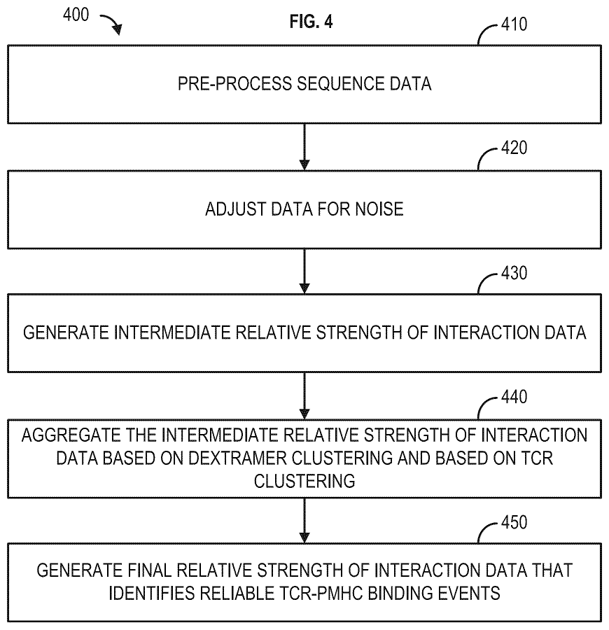

In some aspects, as shown in FIG. 4 the ICONv2 module 108 can be configured to perform a method 400 comprising pre-processing the sequence data at step 410, adjusting data for noise at step 420, generating intermediate relative strength of interaction data at step 430, aggregating the intermediate relative strength of interaction data based on dextramer clustering and based on TCR clustering at step 440, and generating final relative strength of interaction data that identifies reliable TCR-pMHC binding events at step 450. In an embodiment, the ICONv2 process may be performed in a donor, cell, and/or dextramer specific context.

Pre-processing the sequence data at step 410 may comprise generating, populating, and/or modifying one or more data structures. The one or more data structures may comprise one or more arrays and/or matrices. For example, the one or more data structures may comprise one or more dynamic arrays configured to expand to accommodate sequences of varying lengths and/or one or more multidimensional arrays configured for storing and manipulating data that has more than one dimension, such as alignment scores between multiple sequences or expression levels across different conditions.

Pre-processing the sequence data at step 410 may comprise determining and/or receiving RNA sequence data, TCR sequence data, and dextramer sequence data. The RNA sequence data, TCR sequence data, and dextramer sequence data may be determined and/or received by, for example, performing droplet-based single-cell RNA sequencing to generate RNA sequence data, TCR sequence data, and dextramer sequence data for each droplet. The RNA sequence data, TCR sequence data, and dextramer sequence data may be determined and/or received by, for example, downloading or otherwise electronically accessing RNA sequence data, TCR sequence data, and dextramer sequence data for each droplet. The RNA sequence data, the TCR sequence data, and/or the dextramer sequence data may be populated into individual data structures and/or combined into one or more data structures. The RNA sequence data, TCR sequence data, and dextramer sequence data may be categorized as being associated with cell containing droplets or with non-cell containing droplets based on one or more genes present in each droplet.

Pre-processing the sequence data at step 410 may comprise determining one or more clusters for the TCR sequence data and/or the dextramer sequence data. For example, the step 410 may comprise determining, based on a measure of similarity, one or more TCR clusters from the TCR sequence data. For example, the step 410 may comprise determining, based on a measure of similarity, one or more dextramer clusters from the dextramer sequence data. The measure of similarity may be the same measure or may be a different measure. For example, the measure of similarity may be based on sequence similarity.

Clustering TCR sequences based on sequence similarity may involve one or more computational steps that aim to group TCR sequences that share a high degree of similarity into clusters, suggesting that the grouped TCR sequences may recognize the same antigen or have originated from the same ancestral cell. In an embodiment, the TCR sequences may be clustered into clonal groups based on an exact match of amino acids. Each clonal group may thus contain only identical TCR sequences. In an embodiment, TCR sequences may be clustered based on CDR3 region similarity. For example, TCR sequences having identical CDR3 regions may be clustered. In an embodiment, TCR sequences may be clustered based on V and/or J region similarity. For example, TCR sequences having identical V regions may be clustered, TCR sequences having identical J regions may be clustered, and/or TCR sequences having identical V and J regions may be clustered. The TCR sequences may be compared against each other using a suitable sequence alignment method. This may be a global alignment, which compares sequences from end to end, or a more local alignment that looks for the most similar region between two sequences. The choice of alignment method may depend on the specific characteristics of TCR sequences. Commonly used methods for this purpose include BLAST or Smith-Waterman for more detailed alignments. Regions identified as V and/or J regions may be used for alignment of TCR sequences using, for example, igBLAST. ANARCI may also be used to align a given sequence to a database of Hidden Markov Models that describe the germline sequences of antibody and TCR domain types. After alignment, a similarity score may be calculated for each pair of sequences. The similarity score quantifies how similar two sequences are and may be based on the number of matching positions in the alignment, with possible penalties for gaps or mismatches. Similarity scores may be generated from tools such as tcr-dist, GLIPH, TCRVALID, and the like as is known in the art. With the pairwise similarity scores, a distance matrix may be constructed, which may serve as input for a clustering method. A TCR-specific distance matrix may be generated using one of the aforementioned tools. The distance matrix may then be processed using the clustering method such as hierarchical clustering, DBSCAN, or k-means, depending on the desired granularity. Hierarchical clustering may be particularly useful for TCR sequences as it allows for the visualization of clusters in a dendrogram, representing the relationships between sequences. Once the clustering method is applied, it yields groups of TCR sequences that are more similar to each other within the clusters than to sequences outside the cluster. These clusters may then be analyzed further to infer the antigen specificity or to study the clonal expansion of T cells in the context of immune responses.

Clustering dextramer sequences based on sequence similarity may involve one or more computational steps that aim to group dextramer sequences according to homology, which may be indicative of shared functional properties or origins. By way of example, a dextramer may be a complex formed by a cluster of typically 10 monomers, often used in the context of immunology to detect T-cell receptors (TCRs) specific to particular antigens. The dextramer sequences may be aligned using sequence alignment methods. Since dextramers are usually designed to have a high affinity for specific TCRs, local alignment methods like BLAST or Smith-Waterman may be more suitable. These methods allow for the identification of the most similar regions between sequences, which can be useful for accurately determining sequence similarity in the presence of potentially high sequence variability. Similarity scores may then be determined from the alignments, creating a quantitative measure of homology between each pair of dextramer sequences. The similarity scores may be based on the number of identical matches and the nature of any mismatches or gaps found in the aligned dextramer sequences. A distance matrix may be constructed using the similarity scores and provided as input into a clustering method. This distance matrix encapsulates the pairwise distances (or inversely, similarities) between the dextramer sequences. A clustering method is then applied to the distance matrix to group the dextramer sequences into clusters. The distance matrix may then be processed using the clustering method such as hierarchical clustering, DBSCAN, or k-means, depending on the desired granularity. The resultant clusters are sets of dextramer sequences that exhibit a high degree of similarity, suggesting they may bind to similar TCRs or are derived from similar monomers. These clusters can then be subjected to further analysis to elucidate their specificity and affinity to different TCRs or to study the immune response more broadly.

Pre-processing the sequence data at step 410 may comprise generating and/or creating one or more of RNA data, dextramer data, and/or TCR data. The RNA data may comprise data indicating a count of RNA sequences derived from one or more genes present in each cell containing droplet. The dextramer data may comprise data indicating a count of each of one or more dextramers present in each cell containing droplet. The TCR data may comprise data indicating one or more TCR sequences present in each cell containing droplet.

Pre-processing the sequence data at step 410 may comprise identifying each droplet either as a cell containing droplet or as a non-cell containing droplet, i.e., a droplet with no cells in it. Any number of techniques for identifying a droplet as a cell containing droplet or a non-cell containing droplet may be used. In an embodiment, a distribution of UMI counts may be generated and cell barcodes within the same order of magnitude (e.g., barcodes with UMI greater than one tenth of the 99^{th} percentile of UMI in the top N barcodes as ranked by UMI counts) may be considered cell barcodes (e.g., cell containing droplets). Other techniques may be used such as those described, and incorporated by reference herein, in the following: Fleming, et al. Unsupervised removal of systematic background noise from droplet-based single-cell experiments using CellBender. Nat Methods 20, 1323-1335 (2023), Zheng, et al. Massively parallel digital transcriptional profiling of single cells. Nat Commun 8, 14049 (2017), and Lun, A., et al. EmptyDrops: distinguishing cells from empty droplets in droplet-based single-cell RNA sequencing data. Genome Biol 20, 63 (2019).

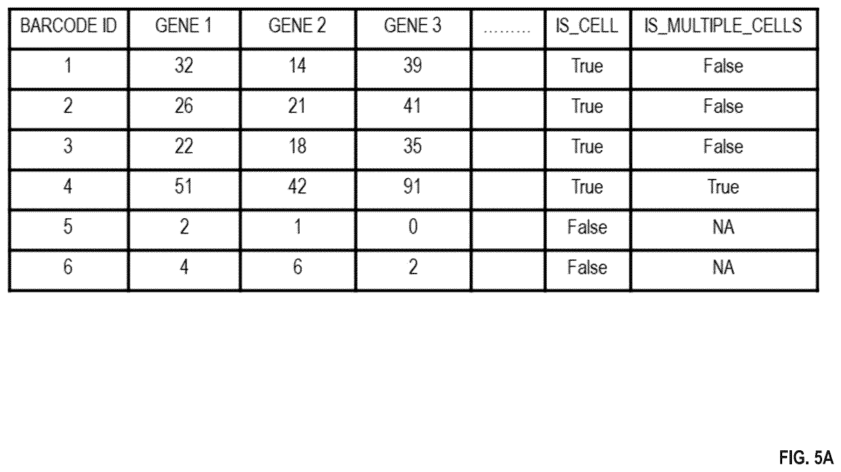

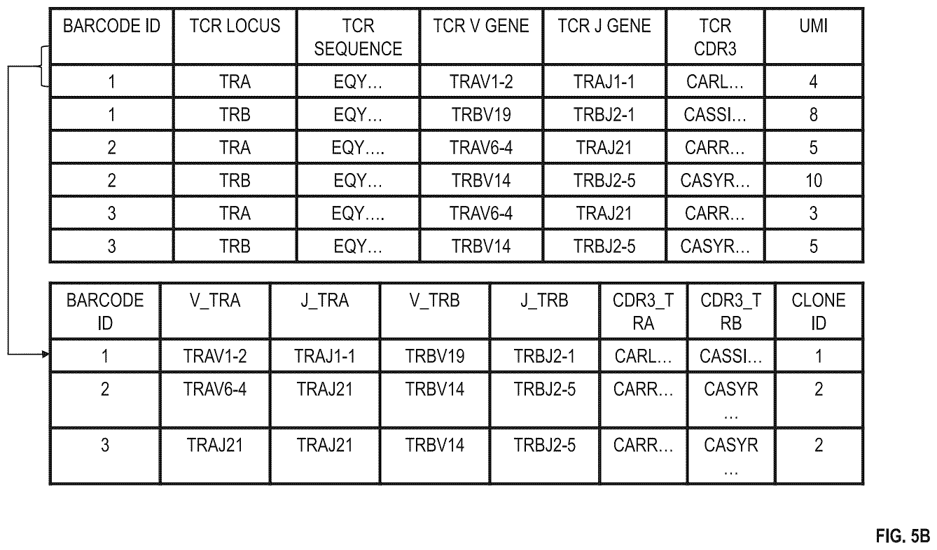

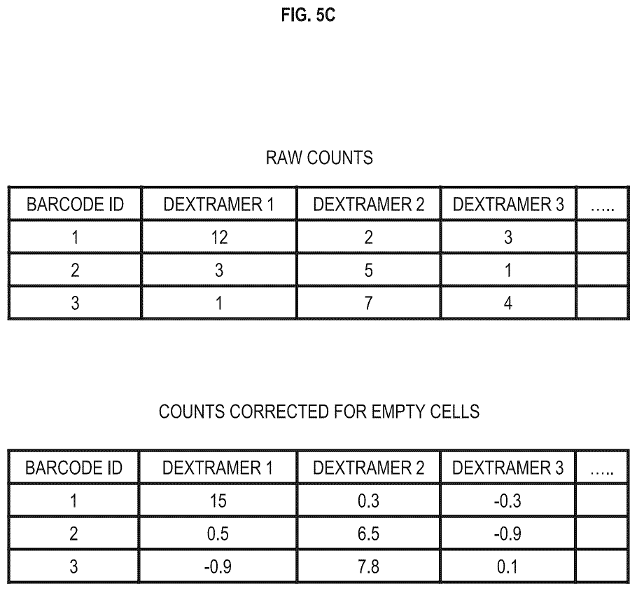

The RNA data may be determined based on mapping the RNA sequence data to a genome reference sequence. For example, the mapping may be used to determine, for each droplet, a count of RNA sequences derived from one or more genes present in each droplet. FIG. 5A shows an example of RNA data.

The TCR data may be determined based on mapping the TCR sequence data to a TCR sequence library. For example, the mapping may be used to identify, for each droplet, one or more TCR sequences present in each droplet. FIG. 5B shows an example of TCR data.

The dextramer data may be determined based on the dextramer sequence data. For example, the dextramer sequence data may be used to determine, for each droplet, a count of each of one or more dextramers present in each droplet. FIG. 5C shows an example of dextramer data.

Pre-processing the sequence data at step 410 may thus result in generation of RNA data, dextramer data, and/or TCR data. The RNA data, the dextramer data, and/or the TCR data may be contained within one or more data structures.

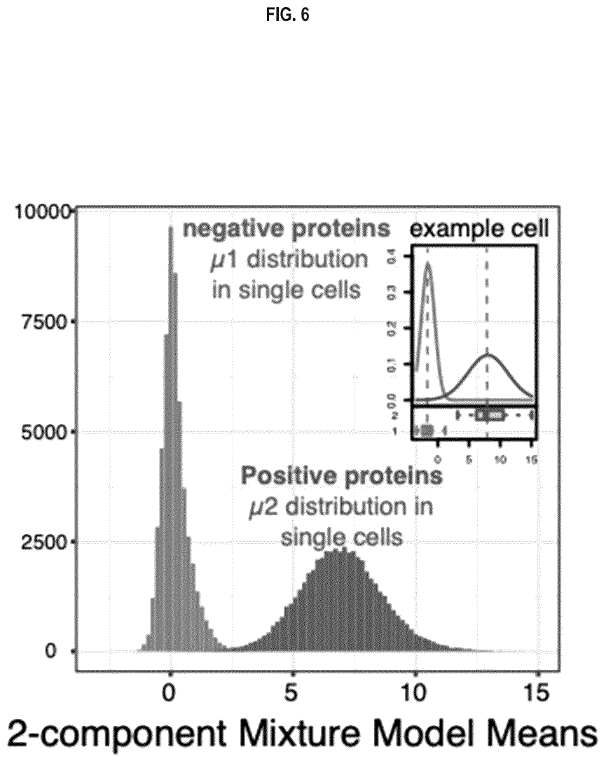

Adjusting data for noise at step 420 may comprise determining a background correction derived from counts of dextramers present in non-cell containing droplets and adjusting the dextramer data based on the background correction. Determining a background correction and adjusting the dextramer data based on the background correction may comprise using non-cell droplets to z-scale the cell-containing droplets. The result is that the cell containing droplets have a larger z-score than the non-cell containing droplets, as the cell containing droplets have a much higher signal. In some instances, one or more cell-containing droplets may have little signal and therefore have a relatively lower z-score. In an embodiment, DSB normalization may be applied to the dextramer data. In an embodiment, the methods taught for normalizing and denoising protein expression data as taught by Mulè, M. P., et al., Normalizing and denoising protein expression data from droplet-based single cell profiling. Nat Commun 13, 2099 (2022), incorporated herein by reference, may be adapted for normalizing and denoising dextramer data. For example, empty droplets (no cell) and full droplets (at least one cell) may be used to define two modes in a 2-component mixture model as shown in FIG. 6. The dextramer data for droplets without cells may be then used to understand the background distribution and the disclosed methods may remove the background distribution from the cell-containing dextramer data.

Adjusting data for noise at step 420 may comprise determining data in the dextramer data associated with a droplet comprising two or more cells and data associated with a droplet containing no TCR sequence. The RNA data may be used to determine the data in the dextramer data associated with a droplet comprising two or more cells and data associated with a droplet containing no TCR sequence. Data in the dextramer data associated with a droplet comprising two or more cells and data associated with a droplet containing no TCR sequence may be removed from the dextramer data. For example, a T cell with an unexpectedly high number of genes (e.g., >2500 genes per cell, or some other predetermined threshold) may be classified as a doublet. A T cell with a high fraction of mitochondrial gene expression (e.g., ratio of mitochondrial gene expression to the total gene expression >0.2, or some other predetermined threshold) may be classified as a dead cell (no TCR sequence). A T cell with too few genes detected (<200 genes per cell, or some other predetermined threshold) may be classified as a dead cell (no TCR sequence).

Adjusting data for noise at step 420 may comprise normalizing the TCR data. In an embodiment, normalizing the TCR data may comprise dropping from further consideration any droplet that does not contain both an alpha chain and a beta chain. In an embodiment, normalizing the TCR data may comprise dropping from further consideration any droplet that does not contain either an alpha chain or a beta chain.

Adjusting data for noise at step 420 may comprise normalizing the dextramer data. Normalizing the dextramer data may comprise normalizing counts of dextramers in the dextramer data on a per dextramer basis. In an embodiment, dextramer data may comprise a table of droplet data and dextramer data. The table may have table entries with counts reflecting the corrected number of dextramers in each droplet (the counts are corrected for background noise by adjusting for empty droplet counts (non-cell containing droplets)). Thus, each dextramer may be associated with a list of counts (e.g., a dextramer column). It is expected that most cells (droplets) will have little to no signal for a dextramer, with a small number of cells having a higher signal. Such data may be visualized as a histogram with counts on the x axis. A peak will exist near 0 and there will be a secondary higher peak. The location and breadth of the higher peak, as well as how far the higher peak is from the zero peak, will be different for different dextramers and experiments. Since the different dextramers have positive peaks occurring at different absolute values, the x axis (counts) can be scaled such that the positive peak occurs near 1.

Normalizing the dextramer data, per dextramer, may thus comprise determining a threshold that separates the histogram (per dextramer) into two categories. One or more existing techniques may be used, such as minimum threshold, Otsu, and the like. Should the technique fail to find a “good” solution (e.g., does not converge), then a “best-guess” (chosen for all dextramers) may be determined by an expert review of the data. If the technique used determined that the threshold is below this best-guess, then the threshold may be set to the best-guess. The determined threshold (1) may be used to determine a second parameter sigma:

$$σ = ( \frac{{\sum }_{x ϵ X} x 𝕀 ( x > t )}{\vert {\sum }_{x ϵ X} 𝕀 ( x > t ) \vert} ) - t$$

where I is the indicator function, X is the dextramer signal for dextramer under study for one cell, X is the set of dextramer signal for dextramer under study to all cells, t is the determined threshold. This represents the mean of the counts higher than the threshold, with the threshold subtracted. To apply scaling correction to data for one dextramer,

$${x}^{′} = \frac{x - t}{σ}$$

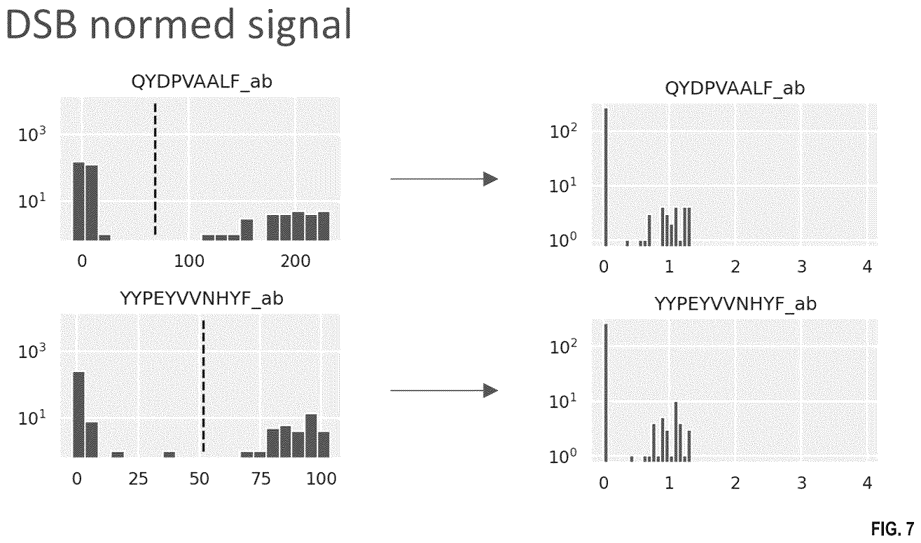

may be applied. This now means that x at the threshold is 0; x below threshold is less than 0; and when x equals the mean of the counts greater than the threshold, the scaled counts will be 1. The values of x may then be clipped. Any x greater than predetermined value (e.g., 4) may be set to the predetermined value. Any x less than an epsilon may be set to the epsilon. Epsilon may be zero but may cause computational divide by zero errors. As shown in FIG. 7, background subtraction and normalization may be used to solve both “sticky” dextramer issues. A separatrix of negative and positive signal may be defined and data may be scaled such that a positive peak is 1 (mean), and the remaining are set to 0.

Thus, for each peptide, the signal is background corrected and normalized to the same scale. The noise may be filtered from the dextramer data by determining a value RC that measures specificity of a cell-dextramer pair according to equation 1. RC assesses, in a cell, the fractional UMI content to each dextramer.

$${RC}_{cell , dex} = \frac{{x}_{c ell , dex}}{{\sum }_{dex ′} {x}_{cell , dex ′}} \qquad ( 1 )$$

Then, a value of RT may be determined according to equation 2. RT measures how fractionally often cells of the same clone are found binding the same dextramer.

$${RT}_{clonotype , dex} = \frac{{y}_{clono , dex}}{{\sum }_{dex ′} {y}_{clono , dex ′}} \qquad ( 2 )$$

Then, average RC (meanRC) for each dextramer over the cells in the clonotype may be determined according to equation 3.

$${y}_{clono , dex} = \frac{1}{n\_clono} {\sum }_{cell ∈ clono} {RC}_{cell , dex} \qquad ( 3 )$$

Data associated with cells that are “not specific” may be removed before calculating meanRC so that “unbound” or “messy” cells do not contribute to RT. RT is a measure of how consistent a clonotype is and poor quality cells should not be included in this metric. meanRC also introduces a free parameter—maxRC in cell to be allowed into the meanRC calculation. In an embodiment, the threshold may be set to max (RC)>0.8.

The method 400 may comprise generating intermediate relative strength RC,

$$\frac{{x}_{cell , dex}}{{\sum }_{dex ′} {x}_{c ell , dex ′}} ,$$

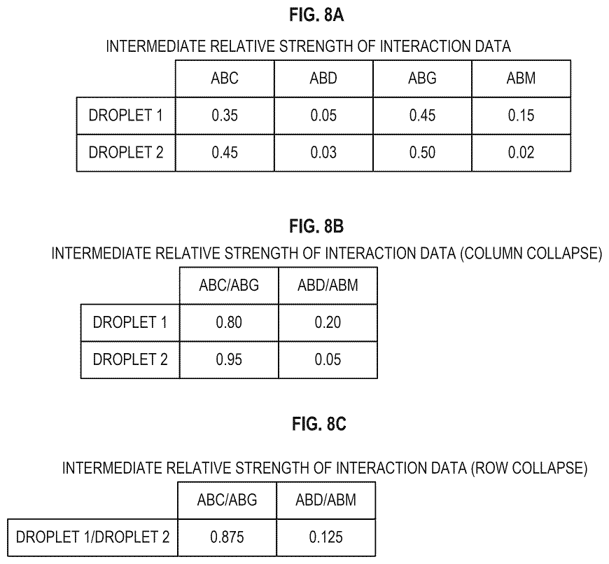

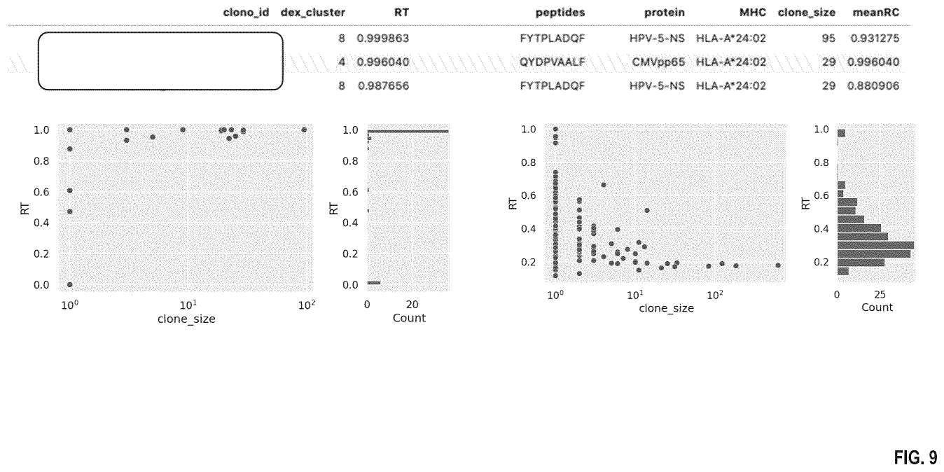

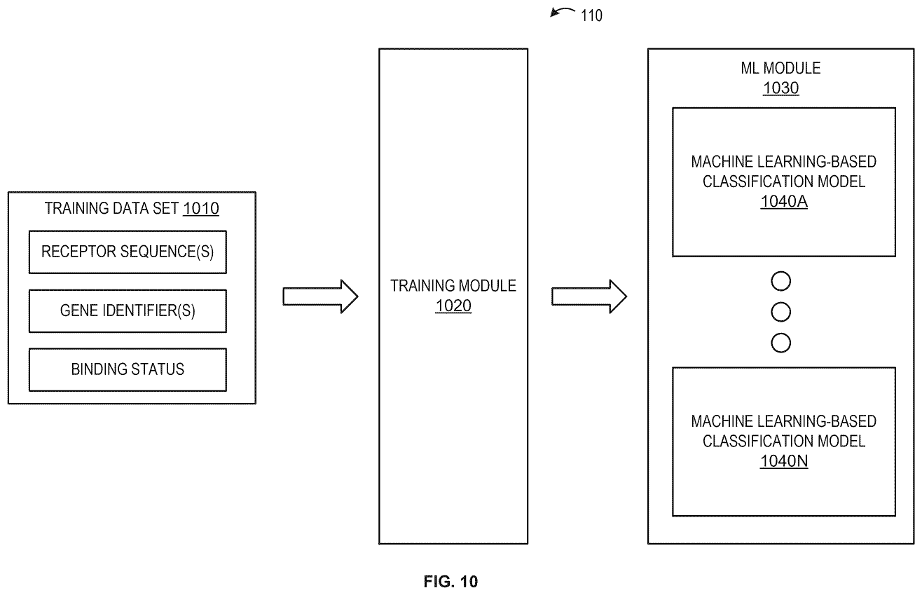

of interaction data at step 430. Generating intermediate relative strength of interaction data at step 430 may comprise generating, based on the dextramer data, intermediate relative strength of interaction data indicating a strength of interaction for a TCR with each of one or more dextramers and removing, from the intermediate relative strength of interaction data, data that does not satisfy a threshold. The threshold may be, for example, zero or some other predetermined, non-zero threshold. Removing, from the intermediate relative strength of interaction data, data that does not satisfy a threshold of zero will remove cells with no signal. For example, if all dextramers in a cell had 0 scaled signal, then data associated with this cell is removed. FIG. 8A shows an example of intermediate strength of interaction data. TCR(s) in Droplet 1 have an intermediate strength of interaction of 0.35 with dextramer ABC, 0.05 with dextramer ABD, 0.45 with dextramer ABG, and 0.15 with dextramer ABM. TCR(s) in Droplet 1 have an intermediate strength of interaction of 0.45 with dextramer ABC, 0.03 with dextramer ABD, 0.50 with dextramer ABG, and 0.02 with dextramer ABM.

Aggregating the intermediate relative strength of interaction data at step 440 may comprise data aggregation according to dextramer cluster. Data aggregation according to dextramer cluster may comprise aggregating, e.g., by taking the mean of the RC values within each dextramer cluster for each cell, based on the one or more dextramer clusters, data of the intermediate relative strength of interaction data and removing, from the intermediate relative strength of interaction data, data that does not satisfy an interaction threshold (s). Because of having aggregated over the clusters, data may be removed for cells that have appreciable signal across multiple clusters of dextramer, which would be unexpected. The interaction threshold (s) may be, for example, a maximal value of RC (max(RC)) and/or an entropy value of RC (entropy(RC)), calculated per cell. In an embodiment, the interaction threshold (s) may be a max(RC) threshold and data associated with cells having a max(RC) below the max(RC) threshold may be removed. The max(RC) threshold may be selected based on a number of dextramer clusters and/or a user preference for noise. By way of example, the max(RC) threshold may range from about 0.1 to about 0.8. In an embodiment, the interaction threshold (s) may be an entropy(RC) threshold and data associated with cells having a entropy(RC) below the entropy(RC) threshold may be removed. Use of logarithm in base (number of dextramer clusters) may generate an entropy value for each cell and may be between 0 and 1. The result will be 0 for cells that only have signal to one dextramer cluster or 1 for cells that have equal signal to all clusters. An entropy(RC) threshold may then be selected and may be between 0 and 1, depending on user preference and/or noise preference. For example, data may be removed for cells with entropy (base of number of dex clusters) greater than about 0.5.

Aggregating, based on the one or more dextramer clusters, data of the intermediate relative strength of interaction data at step 440 may comprise collapsing columns of a data structure containing the intermediate relative strength of interaction data. FIG. 8B shows a result of aggregating, based on the one or more dextramer clusters, data of the intermediate relative strength of interaction data. By way of example, as shown in FIG. 8B, dextramers ABC and ABG may be clustered and ABD and ABM may be clustered. TCR(s) in Droplet 1 have an intermediate relative strength of interaction of 0.80 (0.35+0.45) with dextramer cluster ABC/ABG and 0.20 (0.05+0.15) with dextramer cluster ABD/ABM. TCR(s) in Droplet 2 have an intermediate relative strength of interaction of 0.95 (0.45+0.50) with dextramer cluster ABC/ABG and 0.05 (0.03+0.02) with dextramer cluster ABD/ABM.

Generating intermediate relative strength of interaction data at step 430 may comprise data aggregation according to TCR cluster. Data aggregation according to TCR cluster may comprise aggregating, based on the one or more TCR clusters, data of the intermediate relative strength of interaction data, e.g., via mean aggregation, and removing, from the intermediate relative strength of interaction data, data that does not satisfy an clonal specificity threshold (p). The clonal specificity threshold (p) may be, for example, 0.6. Aggregating, based on the one or more TCR clusters, data of the intermediate relative strength of interaction data may comprise aggregating RC values over cells with the same TCR (e.g., TCR cluster). An RT,

$$\frac{{y}_{clono , dex}}{{\sum }_{dex ′} {y}_{c l ono , dex ′}} ,$$

value may be determined for each TCR cluster, representing the, e.g. mean, fractional aggregated signal to each dextramer group within each TCR cluster. A TCR cluster is expected to bind one dextramer group strongly: that is, the largest fractional signal to a dextramer group for one TCR group is expected to be close to 1. Accordingly, for each TCR cluster, data associated with the dextramer cluster with highest RT may be retained. A list of TCR clusters may be generated with the their highest associated dextramer cluster and the value of RT to that dextramer cluster. The clonal specificity threshold (p) may be applied to the list such that data associated with TCR clusters having an RT below the clonal specificity threshold (p) are discarded. The clonal specificity threshold (p) may be, for example, about 0.6, wherein data associated with TCR clusters are discarded if ˜<60% of signal was for its strongest dextramer association). One may also choose to remove interaction based on the number of TCRs are in a cluster, e.g., requiring at least for example 3 TCRs. Alternatively, one may choose a sliding scale of clonal specificity threshold (p) which is larger for TCR clusters with fewer TCRs and lower for TCR clusters with more TCRs.

Aggregating, based on the one or more TCR clusters, data of the intermediate relative strength of interaction data may comprise collapsing rows of a data structure containing the intermediate relative strength of interaction data. FIG. 8C shows a result of aggregating, based on the one or more TCR clusters, data of the intermediate relative strength of interaction data. By way of example, as shown in FIG. 8B, TCRs in Droplet 1and Droplet 2 may be clustered. The cluster of TCRs in Droplet 1/Droplet 2 has an intermediate relative strength of interaction of

$$0.875 ( \frac{0.80 + 0.95}{2} )$$

with dextramer cluster ABC/ABG and

$$0.125 ( \frac{0.20 + 0.05}{2} )$$

with dextramer cluster ABD/ABM.

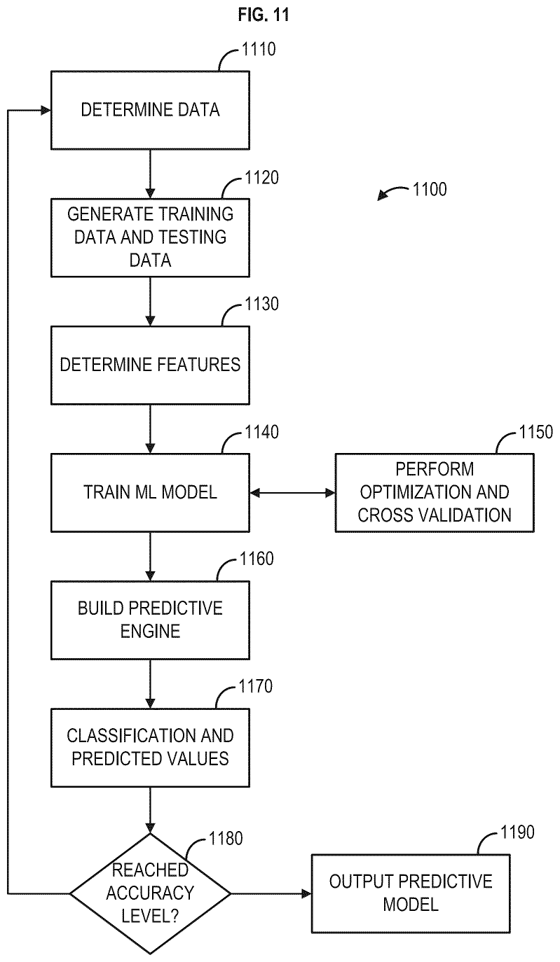

The method 400 may comprise generating final relative strength of interaction data that identifies reliable TCR-pMHC binding events at step 450. Generating final strength of interaction data at step 450 may comprise creating final strength of interaction data comprising data remaining in the intermediate relative strength of interaction data that indicates a strength of interaction for a TCR with each of one or more dextramer clusters and data remaining in the intermediate relative strength of interaction data that indicates a strength of interaction for a TCR cluster with each of one or more dextramer clusters. FIG. 9 shows an example of final strength of interaction data. The final strength of interaction data may be output. The final strength of interaction data represents reliable TCR-pMHC binding events. Such data may be considered at least a portion of a training data set for use in a machine learning process. Such training data may be provided to the predictive module 110.

### C. Methods of Using Reliable Receptor-pMHC Binding for Machine Learning

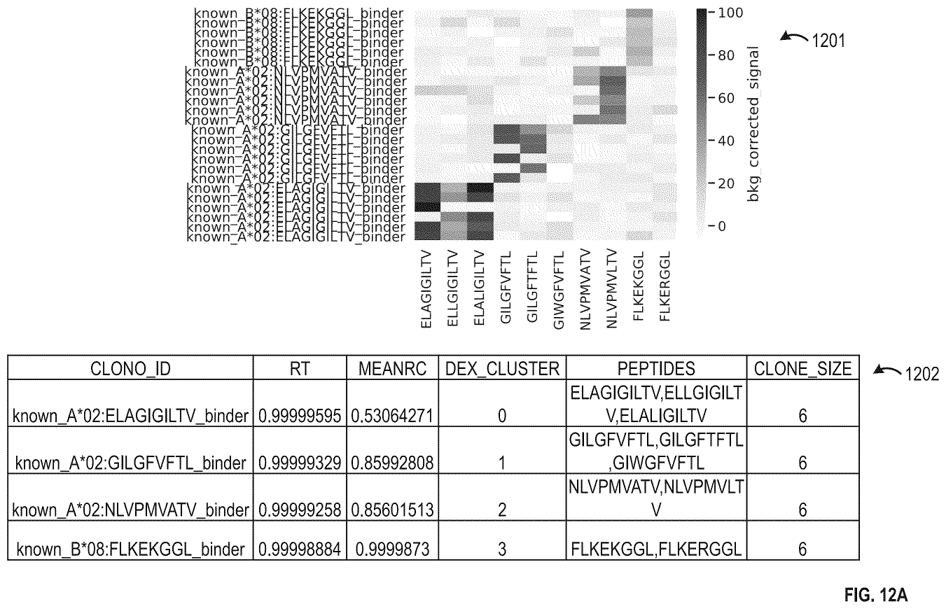

Turning now to FIG. 10, the predictive module 110 is described. The predictive module 110 may be configured to use machine learning (“ML”) techniques to train, based on an analysis of one or more training data sets 1010 by a training module 1020, at least one ML module 1030 that is configured to predict a binding affinity for a given receptor sequence.

The training data set 1010 may comprise one or more receptor sequences, one or more gene identifiers, a binding status, and an identifier of a peptide to which the receptor sequence bound (if any). The binding status may indicate “yes” for a receptor sequence that bound to a peptide or “no” for a receptor sequence that did not bind to a peptide. For receptor sequences that bound to a peptide, the identifier of the peptide can be used to identify an antigen associated with the peptide. Such data may be derived in whole or in part from the sequence data 104 processed by the ICONv2 module 108. In an embodiment, TCR-CDR3 amino acid sequences may be determined from the sequence data 104, including associated V, D, and J gene identifiers, a label indicating binding status (Yes, No), and an identifier of a peptide to which the TCR-CDR3 amino acid sequences bound. The TCR-CDR3 amino acid sequences may be encoded into numbers to represent the 20 possible amino acids. Padding may be applied to sequences as needed. The V and J gene identifiers may be one-hot encoded to provide a categorical and discrete representation of the gene identifiers in numerical space. The encoded TCR-CDR3 amino acid and V and J gene identifiers may be concatenated together to represent one TCR record and associated with the label indicating binding status (Yes, No). The label may further indicate the specific peptide to which the TCR bound. One or more TCR records may be combined to result in the training data set 1010.

A subset of the TCR records may be randomly assigned to the training data set 1010 or to a testing data set. In some implementations, the assignment of data to a training data set or a testing data set may not be completely random. In this case, one or more criteria may be used during the assignment. In general, any suitable method may be used to assign the data to the training or testing data sets, while ensuring that the distributions of yes and no labels are somewhat similar in the training data set and the testing data set.

The training module 1020 may train the ML module 1030 by extracting a feature set from a plurality of TCR records (e.g., labeled as yes) in the training data set 1010 according to one or more feature selection techniques. The training module 1020 may train the ML module 1030 by extracting a feature set from the training data set 1010 that includes statistically significant features of positive examples (e.g., labeled as being yes) and statistically significant features of negative examples (e.g., labeled as being no).

The training module 1020 may extract a feature set from the training data set 410 in a variety of ways. The training module 1020 may perform feature extraction multiple times, each time using a different feature-extraction technique. In an example, the feature sets generated using the different techniques may each be used to generate different machine learning-based classification models 1040. For example, the feature set with the highest quality metrics may be selected for use in training. The training module 1020 may use the feature set(s) to build one or more machine learning-based classification models 1040A-1040N that are configured to indicate whether a new receptor sequence (e.g., with an unknown binding status) is likely or not likely to bind to a peptide or pMHC.

The training data set 1010 may be analyzed to determine any dependencies, associations, and/or correlations between features and the yes/no labels in the training data set 1010. The identified correlations may have the form of a list of features that are associated with different yes/no labels. The term “feature,” as used herein, may refer to any characteristic of an item of data that may be used to determine whether the item of data falls within one or more specific categories. By way of example, the features described herein may comprise one or more sequence patterns, amino acid sequences of one or both alpha and beta chains, names of v and j gene segments of one or both alpha and beta chains.

A feature selection technique may comprise one or more feature selection rules. The one or more feature selection rules may comprise an feature occurrence rule. The feature occurrence rule may comprise determining which features in the training data set 410 occur over a threshold number of times and identifying those features that satisfy the threshold as candidate features.

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training data set 1010 to generate a first list of features. A final list of candidate features may be analyzed according to additional feature selection techniques to determine one or more candidate feature groups (e.g., groups of features that may be used to predict binding). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning methods. Instead, features may be selected on the basis of scores in various statistical tests for their correlation with the outcome variable (e.g., yes/no).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more candidate feature groups. Forward feature selection is an iterative method that begins with no feature in the machine learning model. In each iteration, the feature which best improves the model is added until an addition of a new variable does not improve the performance of the machine learning model. As an example, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization method which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 1020 has generated a feature set(s), the training module 420 may generate a machine learning-based classification model 1040 based on the feature set(s). A machine learning-based classification model may refer to a complex mathematical model for data classification that is generated using machine-learning techniques. In one example, the machine learning-based classification model 1040 may include a map of support vectors that represent boundary features. By way of example, boundary features may be selected from, and/or represent the highest-ranked features in, a feature set.

The training module 1020 may use the feature sets extracted from the training data set 1010 to build a machine learning-based classification model 1040A-1040N for each classification category (e.g., yes, no). In some examples, the machine learning-based classification models 1040A-1040N may be combined into a single machine learning-based classification model 1040. Similarly, the ML module 1030 may represent a single classifier containing a single or a plurality of machine learning-based classification models 1040 and/or multiple classifiers containing a single or a plurality of machine learning-based classification models 1040.

The extracted features (e.g., one or more candidate features) may be combined in a classification model trained using a machine learning approach such as discriminant analysis; decision tree; a nearest neighbor (NN) method (e.g., k-NN models, replicator NN models, etc.); statistical method (e.g., Bayesian networks, etc.); clustering method (e.g., k-means, mean-shift, etc.); neural networks (e.g., reservoir networks, artificial neural networks, etc.); support vector machines (SVMs); logistic regression methods; linear regression methods; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting ML module 1030 may comprise a decision rule or a mapping for each candidate feature to assign an binding status to a new receptor sequence.

In an embodiment, the training module 1020 may train the machine learning-based classification models 1040 as a convolutional neural network (CNN). The CNN may comprise at least one convolutional feature layer and three fully connected layers leading to a final classification layer (softmax). The final classification layer may finally be applied to combine the outputs of the fully connected layers using softmax functions as is known in the art.

The candidate feature(s) and the ML module 1030 may be used to predict the binding statuses (and associated peptides) of a plurality of TCR records in the testing data set. In one example, the result for each TCR record includes a confidence level that corresponds to a likelihood or a probability that the receptor sequence will bind to a peptide. The confidence level may be a value between zero and one, and it may represent a likelihood that the receptor sequence belongs to a yes/no binding status with regard to one or more peptides. In one example, when there are two statuses (e.g., yes and no), the confidence level may correspond to a value p, which refers to a likelihood that a particular receptor sequence belongs to the first status (e.g., yes). In this case, the value 1−p may refer to a likelihood that the particular receptor sequence belongs to the second status (e.g., no). In general, multiple confidence levels may be provided for each test receptor sequence and for each candidate feature when there are more than two statuses. A top performing candidate feature may be determined by comparing the result obtained for each test receptor sequence with the known yes/no binding status for each test receptor sequence. In general, the top performing candidate feature will have results that closely match the known yes/no binding statuses.

The top performing candidate feature(s) may be used to predict the yes/no binding status of a receptor sequence with regard to one or more peptides. For example, a new TCR sequence may be determined/received. The new TCR sequence may be provided to the ML module 1030 which may, based on the top performing candidate feature, classify the new TCR sequence as either binding (yes) or not binding (no) and an indication of the binding peptide(s).

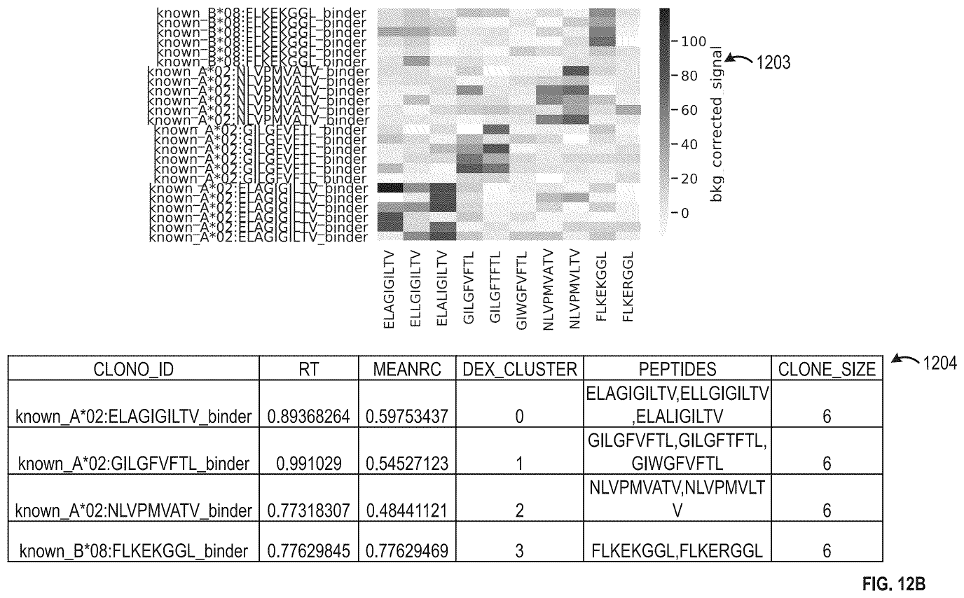

FIG. 11 is a flowchart illustrating an example training method 1100 for generating the ML module 1130 using the training module 1020. The training module 1020 can implement supervised, unsupervised, and/or semi-supervised (e.g., reinforcement based) machine learning-based classification models 1040. The method 1100 illustrated in FIG. 11 is an example of a supervised learning method; variations of this example of training method are discussed below, however, other training methods can be analogously implemented to train unsupervised and/or semi-supervised machine learning models.

The training method 1100 may determine (e.g., access, receive, retrieve, etc.) first sequence data that has been processed by the ICONv2 module 108 at step 1110. The sequence data may comprise a labeled set of receptor sequences. The labels may correspond to binding status (e.g., yes or no) and identification of peptide(s) to which the receptor sequence bound.

The training method 1100 may generate, at step 1120, a training data set and a testing data set. The training data set and the testing data set may be generated by randomly assigning labeled receptor sequences to either the training data set or the testing data set. In some implementations, the assignment of labeled receptor sequences as training or testing samples may not be completely random. As an example, a majority of the labeled receptor sequences may be used to generate the training data set. For example, 75% of the labeled receptor sequences may be used to generate the training data set and 25% may be used to generate the testing data set.

The training method 1100 may determine (e.g., extract, select, etc.), at step 1130, one or more features that can be used by, for example, a classifier to differentiate among different classification of binding status (e.g., yes vs. no) with regard to one or more peptides. As an example, the training method 1100 may determine a set features from the labeled receptor sequences. In a further example, a set of features may be determined from labeled receptor sequences different than the labeled receptor sequences in either the training data set or the testing data set. In other words, labeled receptor sequences may be used for feature determination, rather than for training a machine learning model. Such labeled receptor sequences may be used to determine an initial set of features, which may be further reduced using the training data set.

The training method 1100 may train one or more machine learning models using the one or more features at step 1140. In one example, the machine learning models may be trained using supervised learning. In another example, other machine learning techniques may be employed, including unsupervised learning and semi-supervised. The machine learning models trained at 1140 may be selected based on different criteria depending on the problem to be solved and/or data available in the training data set. For example, machine learning classifiers can suffer from different degrees of bias. Accordingly, more than one machine learning model can be trained at 1140, optimized, improved, and cross-validated at step 1150.

The training method 1100 may select one or more machine learning models to build a predictive model at 1160. The predictive model may be evaluated using the testing data set. The predictive model may analyze the testing data set and generate predicted binding statuses at step 1170. Predicted binding statuses may be evaluated at step 1180 to determine whether such values have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a receptor sequence as binding that was in reality not binding. Conversely, the false negatives of the predictive model may refer to a number of times the machine learning model classified a receptor sequence as not binding when, in fact, the receptor sequence was binding. True negatives and true positives may refer to a number of times the predictive model correctly classified one or more receptor sequences as binding or non-binding. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 1030) may be output at step 1190; when the desired accuracy level is not reached, however, then a subsequent iteration of the training method 1100 may be performed starting at step 1110 with variations such as, for example, considering a larger collection of sequence data.

In an embodiment, unsupervised TCR clustering may be used to cluster TCRs present in one or more ICONv2 processed datasets with other TCR data that may be derived from the same or different individuals. Unsupervised TCR clustering may be performed using clustering algorithms (such as e.g., Density-Based Spatial Clustering of Applications with Noise (DBSCAN)) on devised distance metrics between TCRs (e.g., tcr-dist) or distance metrics themselves learned in an unsupervised fashion (e.g., distance in a Variational AutoEncoder (VAE) latent space, distance in a large-language-model embedding space). ter-dist may be used as a specific metric for measuring distances (similarities/differences) between TCRs. VAEs are a type of neural network used for generating complex models that create a latent space where similar data points are closer to each other. Distance in this space can signify similarity between TCRs. In Large-Language-Model Embedding Space, TCR data may be represented in a high-dimensional space similar to how words are represented in language models. Distances in this space may indicate similarity.

In this manner, TCRs from assays that do not have dextramer associations that co-cluster with TCRs in the ICONv2 processed data may be assigned the same antigen reactivity as those TCRs from the ICONv2 processed TCRs. This may be particularly useful when the number of TCRs with ICONv2 labels to a single dextramer are low in number, e.g. <5. Alternatively, or additionally, classification of TCR reactivity to pMHC with only few examples in the training data, or not in the training dataset as assigned by ICONv2, may be undertaken by few-shot or zero-shot learning respectively. (see e.g., PanPep, ERGO2, DLpTCR, pMTnet).

Such clustering of TCRs (ICONv2 processed and other) into clusters of TCRs may be used to identify multiple “modes/clusters” of TCR engagement with a single antigen (pMHC) as assigned to some fraction of the clustered TCR data by ICONv2. Assigning a subject's (or sample's) relative use of these different “modes” to an antigen may be used in quantifying and understanding different immune responses between subjects/samples.

### D. Methods of Use

In an aspect, the trained predictive model (e.g., machine learning classifier) may be used to predict a binding status of a TCR sequence with regard to one or more peptides. A TCR sequence may be presented to the machine learning classifier. The machine learning classifier may predict a likelihood that the TCR sequence will bind to one or more specific peptides. Similarly, a plurality of TCR sequences may be presented to the machine learning classifier. The machine learning classifier may predict, for each TCR sequence in the plurality of TCR sequences, a likelihood that each TCR sequence will bind to one or more specific peptides. In an aspect, the machine learning classifier can generate a TCR-peptide map as shown in the example output below.

| TCR Sequence | Peptide | Binding Likelihood |
|---|---|---|
| TCR Sequence 1 | Peptide 1 | 99% |
| TCR Sequence 2 | Peptide 6 | 99% |
| TCR Sequence 2 | Peptide 18 | 97.5% |
| TCR Sequence 2 | Peptide 10 | 68% |
| TCR Sequence 3 | Peptide 4 | 88% |
| TCR Sequence 4 | Peptide 24 | 59% |

A TCR-peptide map thus generated may be used to rapidly identify peptides that a subject's TCR sequences are likely to bind to. A biological sample (e.g., blood) may be obtained from a subject, cells isolated, and sequenced. The subject's TCR sequences may be identified and compared to the TCR-peptide map to identify peptides most likely to bind to the subject's TCR sequences.

In some aspects, identifying and evaluating antigen-specific T cells can be used to better understand the activities of drugs in mono-and combination therapy settings, identify features of potent anti-tumor T cells, screen for immunogenic epitopes in a haplotype-relevant manner, develop new vaccine and TCR therapies, and develop peptide binding methods based on TCR sequence features.

In some aspects, disclosed are methods of identifying a subject using binding patterns of the subject's TCRs. For example, blood can be drawn (first blood draw), cells from the blood can be processed via a single cell-based immune profiling platform, and the resulting data can be processed according to the ICONv2 methods described herein. In some aspects, the cells are exposed to a variety of dextramers comprising pMHCs from a wide range of immunogens. After performing an ICONv2 method as described herein, a reliable TCR binding pattern can be determined. In some aspects, a TCR binding pattern represents the specificity of TCRs to the immunogens on the dextramers. Blood can then be drawn at a different time point (days, weeks, months, years later) from the first blood draw (second blood draw). In some aspects, it would be expected that the second blood draw would likely comprise T cells having TCRs with different sequences than what was present in the first blood draw since there are about 10¹⁵ possible TCR sequences, however, the TCR binding pattern is unlikely to change. The cells from the second blood draw can be exposed to the same dextramers as used for the first blood draw and the resulting data analyzed according to an ICONv2 method. Regardless of the different TCR sequences, the binding data of the first blood draw and second blood draw can be compared and used to determine if they are both from the same subject.

In some aspects, disclosed are methods of identifying a subject using machine learning to predict the binding patterns of the subject's TCRs. Reliable TCR binding data can be identified according to an ICONv2 method as described herein. In some aspects, the reliable TCR binding data can be used to train a machine learning classifier as described herein. The trained machine learning classifier can be used to predict specificity TCR binding pattern of a subject. In some aspects, blood can be drawn (first blood draw) and a TCR binding pattern can be predicted using the trained machine learning classifier. Blood can then be drawn at a different time point (days, weeks, months, years later) from the first blood draw (second blood draw). In some aspects, it would be expected that the second blood draw would likely comprise T cells having TCRs with different sequences than what was present in the first blood draw since there are about 10¹⁵ possible TCR sequences, however, the TCR binding pattern is unlikely to change. Regardless of the different TCR sequences, the trained machine learning classifier may be used to predict a second TCR binding pattern using data derived from the second blood draw. It is possible to predict that the second blood draw is from the same subject as the first blood draw based on the TCR signatures.

In some aspects, a TCR or BCR binding pattern can be established using the described methods. In some aspects, having reliable TCR data identified using the methods described herein allows someone, such as a medical professional, to infer the antigenic history or vaccine history of a subject. In some aspects, reliable TCR data identified using the ICONv2 methods described herein allows someone, such as a medical professional, to infer what pathogens a subject has been exposed to or even what countries the subject has visited. For example, the presence of TCR binding data to pathogens only present in Africa can indicate that the subject has been to Africa and been exposed to those pathogens.

In some aspects, reliable TCR data identified using the ICONv2 methods described herein can assess a current immunologic state of a subject. For example, blood can be drawn (first blood draw), cells from the blood can be processed via a single cell-based immune profiling platform, and the resulting data can be processed according to the ICONv2 methods described herein, resulting in TCR binding data. In some aspects, the dextramers used in establishing the TCR binding data comprise tumor specific pMHCs. Thus, once the TCR binding data has been normalized using an ICONv2 method, and reliable TCR binding data is established, the presence of predicted tumor specific TCRs can be determined. For example, the reliable TCR data can be used in the disclosed machine learning (CNN) methods and therefore the blood from the subject can be analyzed for the presence of predicted tumor specific TCRs. Thus, the presence of tumor specific TCRs can result in early detection of cancer before any tumors or cancer symptoms are detected.

In some aspects, disclosed are methods for selecting T cells for T cell-based therapies. In some aspects, training data can be accumulated using the disclosed methods of machine learning classifying. In some aspects, the classifier can assign probabilities of a pMHC binding to each TCR sequence tested. In some aspects, the TCR sequence tested is associated with a T cell, wherein the T cell can be from a primary or secondary cell culture. This avoids needed to perform binding assays on all T cells being tested to determine if each T cell has a TCR specific to the different pMHCs. Instead, the classifier is relied on for determining the probability of TCR-pMHC binding. Those TCRs, and thus T cell comprising that TCR), classified as being highly selective to a specific pMHC can then be used for T cell therapies. In some aspects, T cells identified through the machine learning classifier can provide safer cell therapies than those T cells identified through binding assays because only the most reliable binding data was used to create the training data used to classify the TCRs associated with the T cells selected.

In some aspects, disclosed are methods for immune monitoring. In some aspects, blood can be drawn from a subject undergoing immunotherapy (e.g. vaccine treatment; immune checkpoint treatment), the cells, particularly the T cells, can be classified, based on the training data established in the disclosed machine learning approaches, as having a specificity to the epitope of interest or not. In some aspects, if a T cell is determined to have specificity to an epitope of interest then one can infer that the subject will be or is responsive to the immunotherapy. For example, if the immunotherapy is a vaccine that triggers an immune response to a cancer specific antigen, then T cells obtained from the subject would be classified based on their probability of binding to the cancer specific antigen. If T cells are selected as having a high probability of binding to the cancer specific antigen based on the training data obtained using the single cell immune profiling technology and ICONv2, then the subject would be considered to be a responder to the immunotherapy (e.g. vaccine).

In some aspects, disclosed are methods of TCR epitope mapping using the disclosed methods. In some aspects, TCR epitope mapping is a term that refers to the process of identifying the specific (in some cases the shortest) amino acid sequence of the epitope of a specific antigen that is recognized by T-cell (CD4+ and/or CD8+) receptors, and at the same time has the potential to stimulate a long lasting and a cytotoxic immune response. While performing the disclosed single cell immune profiling platform technology, dextramers can be used wherein all the different epitopes from one or more antigens of interest can be presented on dextramers. In other words, a single dextramer can comprise a pMHC wherein the peptide of the pMHC is a single epitope from one or more antigens of interest and enough dextramers are used so that every epitope of the one or more antigens of interest are present in the pMHC on the dextramers. T cells can be exposed to the dextramers in the disclosed single cell immune profiling platform with the dextramers comprising a single epitope from one or more antigens of interest and wherein enough dextramers are used so that every epitope of the one or more antigens of interest are present in the pMHC on the dextramers. The single cell sequence data, dextramer sequence data, and single cell TCR sequence data obtained from the single cell immune profiling can provide data about the T cells that bound to the different dextramers (e.g. epitopes). The single cell immune profiling data is then processed using ICONv2 as described herein, therefore resulting in binding data for those cells that had the most reliable binding to one or more epitopes of the one or more antigens of interest. In some aspects, machine learning classification of TCRs that bind to the one or more epitopes of the one or more antigens of interest can be used to predict which T cells from a subject might be reactive against a particular antigen (e.g. tumor antigen).

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for generating single cell sequencing data, the kit comprising reagents for single cell immune profiling. In some aspects, the kits can comprise one or more of the disclosed dextramers comprising pMHCs. In some aspects, the kits can comprise next generation sequencing materials. In some aspects, the kits can comprise multi-omics high-throughput binding data comprising one or more of single cell sequence data, dextramer sequence data, and/or single cell receptor sequence data.

### EXAMPLES

The following examples illustrate the present methods and systems as they relate to colorectal cancer detection. The following Examples are not intended to be limiting thereof.

Synthetic dextramer data was generated for four TCR clones and four sets of peptide MHCs. The synthetic dextramer data represents dextramer data after background correction derived from counts of dextramers present in non-cell containing droplets and adjusting the dextramer data based on the background correction. Each TCR clone is associated with one predetermined cluster of peptides. In some cases, a TCR clone may only bind a subset of the peptides in the pre-decided cluster of peptides. The data may be sampled as follows, for each TCR cell in a TCR clone, and for each dextramer in the synthetic panel:
If dextramer, d, is one of the dextramers that the TCR clone, c, has been pre-determined to bind, with some probability, p, generate a number from a Normal distribution (m_d, sigma) or with 1−p draw from Gumbel (0, sigma_gumbel).
If dextramer, d, one of the dextramers that the TCR clone, c, has been pre-determined to bind: draw from Gumbel (0, sigma_gumbel).
The mean of the binding normal distribution can be different for each dextramer simulating effects of variance in signal strength for different dextramer after background correction, and the sigma_gumbel can be varied simulating additional background noise.

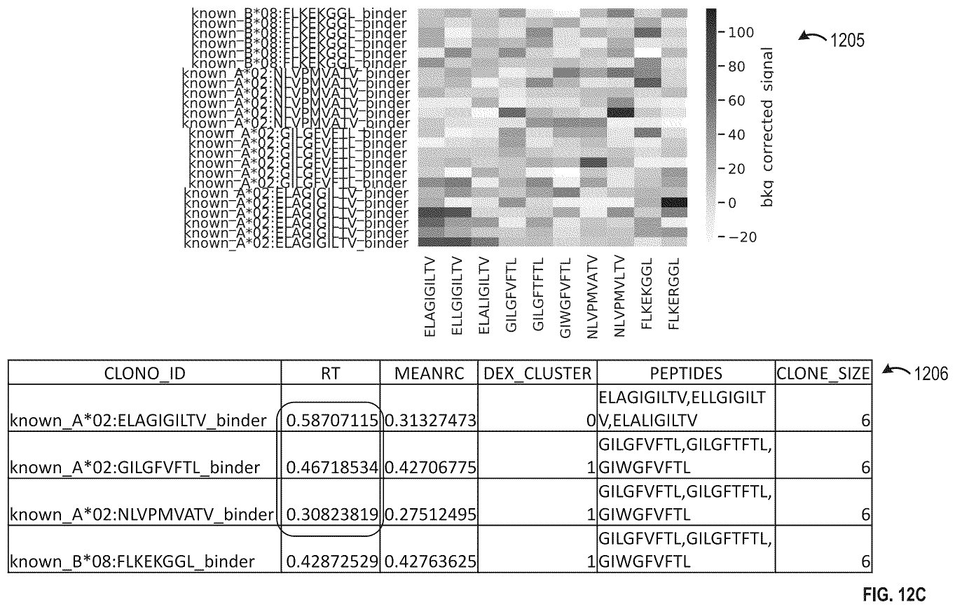

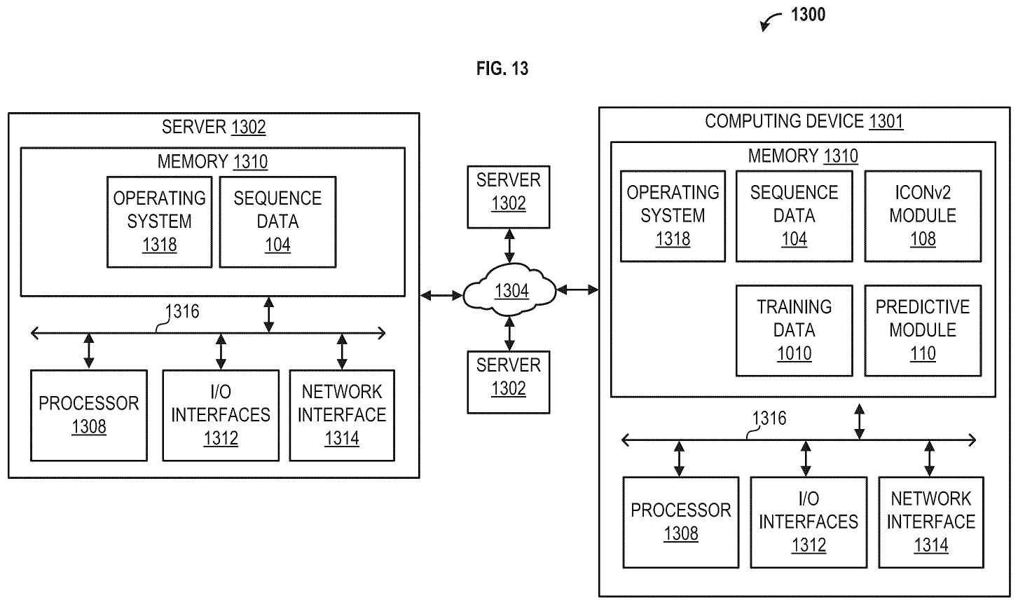

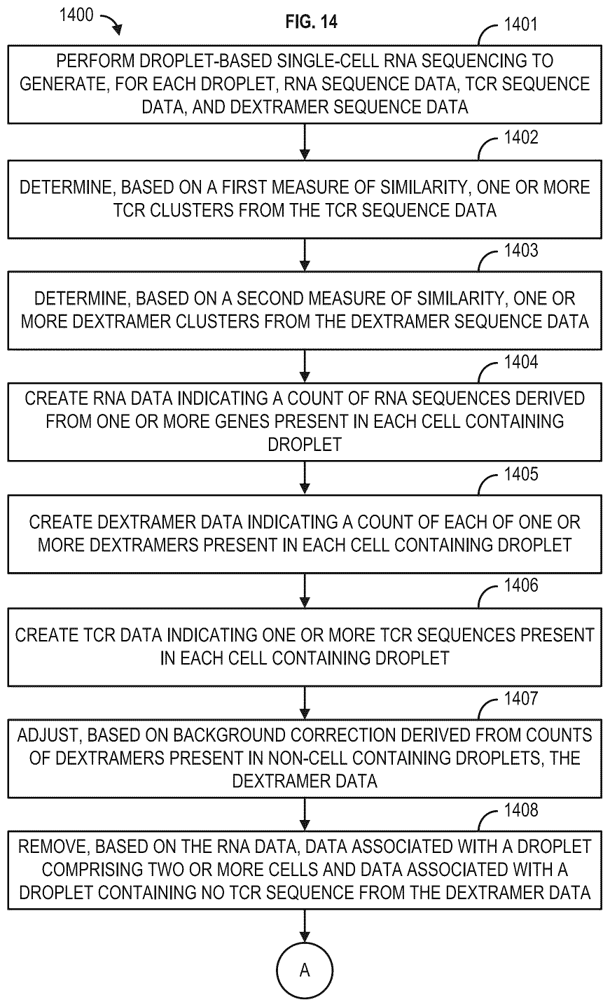

Different scenarios may then be modeled by tuning: p, sigma, and sigma Gumbel. FIG. 12A shows a low noise scenario, including the data input 1201 to the disclosed methods (after background correction) and the example output 1202 of the disclosed methods. Data input 1201 shown in FIG. 12A has cells along the x-axis labeled by their TCR (which can repeat) and along the y axis are different dextramer (some of which are similar and co-cluster), the colored rectangles in the plot show the strength of background corrected signal in the cell/dextramer combinations. In the case of FIG. 12A the background noise is simulated as a low value and FIG. 12A shows that while there is some noise due to the selection criteria as denoted here by p (that makes some true cell-dextramer binding events drop to a Gumbel distributed background), that cells with TCRs binding a specific dextramer bind one or more dextramer in each cluster of similar dextramer. Consequently, performing steps 420, 430, and 450, shown in FIG. 4 leads to strong interaction signal for each unique TCR to each dextramer cluster. FIG. 12B shows a medium noise scenario, including the data input 1203 to the disclosed methods (after background correction) and the example output 1204 of the disclosed methods. In this medium noise scenario the parameters are altered, such that sigma is larger, sigma_gumbel is larger and p is larger, meaning that the strength of binding signal relative to non-binding is weaker in FIG. 12B than FIG. 12A due to non-binding interactions having large noise despite non-binding. Despite this increased noise, methods described herein find strong binding signal (RT) despite weaker meanRC within each TCR cluster (as shown in 1204) and this in part is driven by aggregation over similar dextramer. FIG. 12C shows a high noise scenario, including the data input 1205 to the disclosed methods (after background correction) and the example output 1206 of the disclosed methods. In this case the noise is sufficiently high in the background corrected signal that identifying true interactions is harder such that RT is below 0.6 for all TCR/dextramer-cluster pairs, in this case those TCR/dextramer-clusters with RT>0.45 do pick up correct associations (even though mean RC is low).

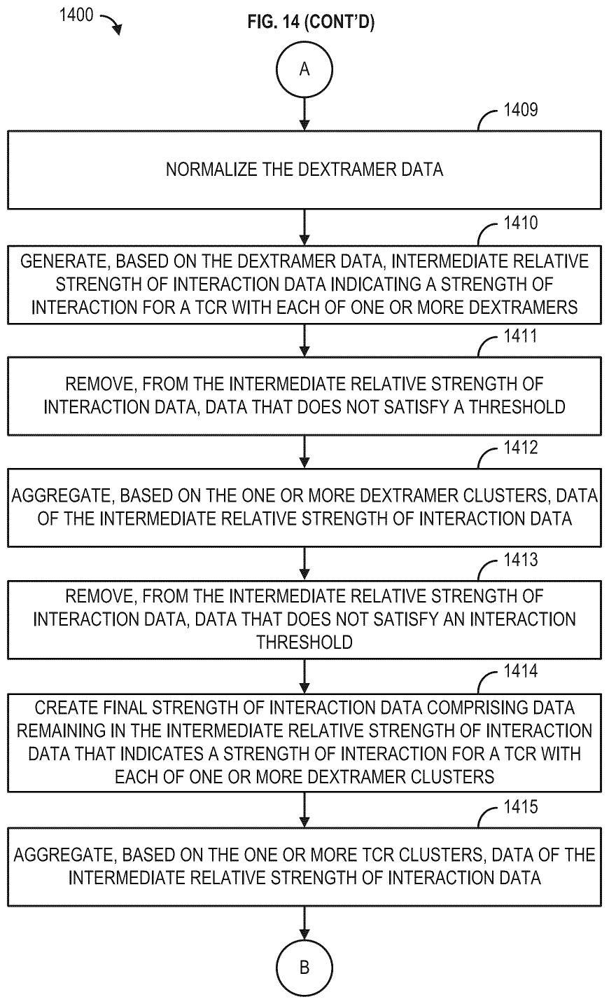

FIG. 13 is a block diagram depicting an environment 1300 comprising non-limiting examples of a computing device 1301 (e.g., the computing device 106) and a server 1302 connected through a network 1304. In an aspect, some or all steps of any described method may be performed on a computing device as described herein. The computing device 1301 can comprise one or multiple computers configured to store one or more of the sequence data 104 (e.g., single cell sequence data, dextramer sequence data, and single cell receptor sequence data), training data 1010 (e.g., labeled receptor sequence data), the ICONv2 module 108, the predictive module 110, and the like. The server 1302 can comprise one or multiple computers configured to store the sequence data 104. Multiple servers 1302 can communicate with the computing device 1301 via the through the network 1304. In an embodiment, the server 1302 may comprise a repository for data generated by the single cell immune profiling platform 102.

The computing device 1301 and the server 1302 can be a digital computer that, in terms of hardware architecture, generally includes a processor 1308, memory system 1310, input/output (I/O) interfaces 1312, and network interfaces 1314. These components (1308, 1310, 1312, and 1314) are communicatively coupled via a local interface 1316. The local interface 1316 can be, for example, but not limited to, one or more buses or other wired or wireless connections, as is known in the art. The local interface 1316 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 1308 can be a hardware device for executing software, particularly that stored in memory system 1310. The processor 1308 can be any custom made or commercially available processor, a central processing unit (CPU), an auxiliary processor among several processors associated with the computing device 1301 and the server 1302, a semiconductor-based microprocessor (in the form of a microchip or chip set), or generally any device for executing software instructions. When the computing device 1301 and/or the server 1302 is in operation, the processor 1308 can be configured to execute software stored within the memory system 1310, to communicate data to and from the memory system 1310, and to generally control operations of the computing device 1301 and the server 1302 pursuant to the software.

The I/O interfaces 1312 can be used to receive user input from, and/or for providing system output to, one or more devices or components. User input can be provided via, for example, a keyboard and/or a mouse. System output can be provided via a display device and a printer (not shown). I/O interfaces 1312 can include, for example, a serial port, a parallel port, a Small Computer System Interface (SCSI), an infrared (IR) interface, a radio frequency (RF) interface, and/or a universal serial bus (USB) interface.

The network interface 1314 can be used to transmit and receive from the computing device 1301 and/or the server 1302 on the network 1304. The network interface 1314 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 1314 may include address, control, and/or data connections to enable appropriate communications on the network 1304.

The memory system 1310 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 1310 may incorporate electronic, magnetic, optical, and/or other types of storage media. Note that the memory system 1310 can have a distributed architecture, where various components are situated remote from one another, but can be accessed by the processor 1308.

The software in memory system 1310 may include one or more software programs, each of which comprises an ordered listing of executable instructions for implementing logical functions. In the example of FIG. 13, the software in the memory system 1310 of the computing device 1301 can comprise the sequence data 104, the training data 1010, the ICONv2 module 108, the predictive module 110, and a suitable operating system (O/S) 1318. In the example of FIG. 13, the software in the memory system 1310 of the server 1302 can comprise, the sequence data 104, and a suitable operating system (O/S) 1318. The operating system 1318 essentially controls the execution of other computer programs and provides scheduling, input-output control, file and data management, memory management, and communication control and related services.

For purposes of illustration, application programs and other executable program components such as the operating system 1318 are illustrated herein as discrete blocks, although it is recognized that such programs and components can reside at various times in different storage components of the computing device 1301 and/or the server 1302. An implementation of the training module 1020 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

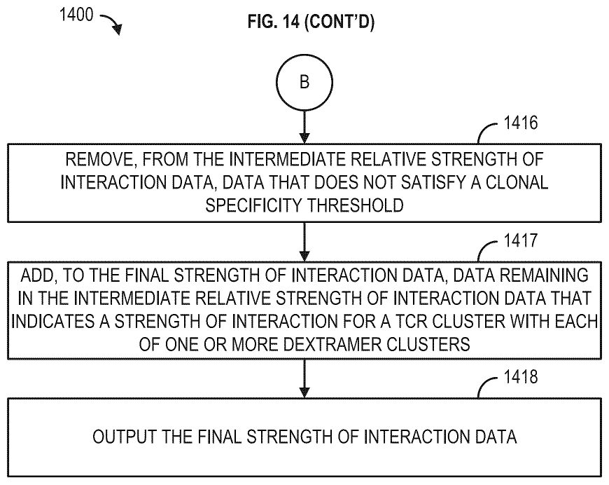

In an embodiment, the ICONv2 module 108 and/or the predictive module 110 may be configured to perform a method 1400, shown in FIG. 14. The method 1400 may be performed in whole or in part by a single computing device, a plurality of electronic devices, and the like. The method 1400 may comprise performing droplet-based single-cell RNA sequencing to generate, for each droplet, RNA sequence data, TCR sequence data, and dextramer sequence data at 1401. The RNA sequence data may comprise sequence data associated with one or more RNA sequences present in a droplet and gene identification data identifying a gene associated with each of the one or more RNA sequences. The TCR sequence data may comprise sequence data associated with one or more TCR sequences present in a droplet. The dextramer sequence data may comprise sequence data associated with one or more dextramer sequences present in a droplet and dextramer identification data identifying a dextramer associated with each of the one or more dextramer sequences.

The method 1400 may further comprise identifying each droplet as a cell containing droplet or a non-cell containing droplet. The method 1400 may further comprise identifying a number of cells present in each cell containing droplet. The method 1400 may further comprise identifying each droplet that contains no TCR sequence.

The method 1400 may comprise determining, based on a first measure of similarity, one or more TCR clusters from the TCR sequence data at 1402. The first measure of similarity may comprise sequence similarity. Determining, based on a first measure of similarity, one or more TCR clusters from the TCR sequence data may comprise determining, based on aligning a plurality of TCR sequences of the TCR sequence data, a plurality of similarity scores associated with the plurality of TCR sequences, generating, based on the plurality of similarity scores, a distance matrix, and generating, based on the distance matrix, the one or more TCR clusters.

The method 1400 may comprise determining, based on a second measure of similarity, one or more dextramer clusters from the dextramer sequence data at 1403. The second measure of similarity may comprise sequence similarity. Determining, based on a second measure of similarity, one or more dextramer clusters from the dextramer sequence data may comprise determining, based on aligning a plurality of dextramer sequences of the dextramer sequence data, a plurality of similarity scores associated with the plurality of dextramer sequences, generating, based on the plurality of similarity scores, a distance matrix, and generating, based on the distance matrix, the one or more dextramer clusters.

The method 1400 may comprise creating RNA data indicating a count of RNA sequences derived from one or more genes present in each cell containing droplet at 1404. Creating RNA data indicating a count of RNA sequences derived from one or more genes present in each cell containing droplet may comprise mapping the RNA sequence data to a genome reference sequence and determining, for each droplet, based on the mapping, the count of RNA sequences derived from one or more genes present in each droplet.

The method 1400 may comprise creating dextramer data indicating a count of each of one or more dextramers present in each cell containing droplet at 1405. Creating dextramer data indicating a count of each of one or more dextramers present in each cell containing droplet may comprise: determining, for each droplet, based on the dextramer sequence data, the count of each of one or more dextramers present in each droplet.

The method 1400 may comprise creating TCR data indicating one or more TCR sequences present in each cell containing droplet at 1406. The TCR data may indicate only one TCR sequence present in each cell containing droplet. Creating TCR data indicating one or more TCR sequences present in each cell containing droplet may comprise mapping the TCR sequence data to a TCR sequence library and determining, for each droplet, based on the mapping, the one or more TCR sequences present in each cell containing droplet.

The method 1400 may comprise adjusting, based on background correction derived from counts of dextramers present in non-cell containing droplets, the dextramer data at 1407. Adjusting, based on background correction derived from counts of dextramers present in non-cell containing droplets, the dextramer data may comprise z-scaling cell containing droplets using the non-cell containing droplets.

The method 1400 may comprise removing, based on the RNA data, data associated with a droplet comprising two or more cells and data associated with a droplet containing no TCR sequence from the dextramer data at 1408. Removing, based on the RNA data, data associated with a droplet comprising two or more cells and data associated with a droplet containing no TCR sequence from the dextramer data may comprise deleting the data associated with a droplet comprising two or more cells and the data associated with a droplet containing no TCR sequence from a data structure containing the dextramer data.

The method 1400 may comprise normalizing the dextramer data at 1409. Normalizing the dextramer data may comprise removing from further consideration data associated with a droplet that does not contain both an alpha chain and a beta chain; or removing from further consideration data associated with a droplet that does not either an alpha chain or a beta chain.

The method 1400 may comprise generating, based on the dextramer data, intermediate relative strength of interaction data indicating a strength of interaction for a TCR with each of one or more dextramers at 1410. Generating, based on the dextramer data, intermediate relative strength of interaction data indicating a strength of interaction for a TCR with each of one or more dextramers may comprise a fractional signal to each dextramer in a droplet.

The method 1400 may comprise removing, from the intermediate relative strength of interaction data, data that does not satisfy a threshold at 1411. The threshold may be zero, and removing, from the intermediate relative strength of interaction data, data that does not satisfy the threshold may comprise deleting the data that does not satisfy the threshold from a data structure containing the dextramer data.

The method 1400 may comprise aggregating, based on the one or more dextramer clusters, data of the intermediate relative strength of interaction data at 1412. Aggregating, based on the one or more dextramer clusters, data of the intermediate relative strength of interaction data may comprise collapsing one or more columns of a data structure containing the dextramer data, wherein the columns are collapsed according to dextramers contained within the same dextramer cluster.

The method 1400 may comprise removing, from the intermediate relative strength of interaction data, data that does not satisfy an interaction threshold(s) at 1413. The interaction threshold may be configured for removal of data associated with cells having appreciable signal across a plurality of clusters of dextramers, and wherein removing, from the intermediate relative strength of interaction data, data that does not satisfy the interaction threshold may comprise deleting the data that does not satisfy the interaction threshold from a data structure containing the dextramer data.

The method 1400 may comprise creating final strength of interaction data comprising data remaining in the intermediate relative strength of interaction data that indicates a strength of interaction for a TCR with each of one or more dextramer clusters at 1414. Creating final strength of interaction data comprising data remaining in the intermediate relative strength of interaction data that indicates a strength of interaction for a TCR with each of one or more dextramer clusters may comprise generating a data structure containing the final strength of interaction data.

The method 1400 may comprise aggregating, based on the one or more TCR clusters, data of the intermediate relative strength of interaction data at 1415. Aggregating, based on the one or more TCR clusters, data of the intermediate relative strength of interaction data may comprise collapsing one or more rows of a data structure containing the dextramer data, wherein the rows are collapsed according to TCRs contained within the same TCR cluster.

The method 1400 may comprise removing, from the intermediate relative strength of interaction data, data that does not satisfy a clonal specificity threshold (p) at 1416. The clonal specificity threshold may be configured for removal of data associated with cells of a TCR cluster having weak interaction with a cluster of dextramers, and wherein removing, from the intermediate relative strength of interaction data, data that does not satisfy the clonal specificity threshold may comprise deleting the data that does not satisfy the clonal specificity threshold from a data structure containing the dextramer data.

The method 1400 may comprise adding, to the final strength of interaction data, data remaining in the intermediate relative strength of interaction data that indicates a strength of interaction for a TCR cluster with each of one or more dextramer clusters at 1417. Adding, to the final strength of interaction data, data remaining in the intermediate relative strength of interaction data that indicates a strength of interaction for a TCR cluster with each of one or more dextramer clusters may comprise updating a data structure containing the final strength of interaction data with the data remaining in the intermediate relative strength of interaction data that indicates a strength of interaction for a TCR cluster with each of one or more dextramer clusters.

The method 1400 may comprise outputting the final strength of interaction data at 1418. Outputting the final strength of interaction data may comprise displaying the final strength of interaction data on an output device.

The method 1400 may further comprise training a predictive model based on the final strength of interaction data. The method 1400 may further comprise predicting a binding status of a newly presented TCR sequence according to the trained predictive model. The method 1400 may further comprise presenting, to the predictive model, subject TCR sequence data, determining, by the predictive model, based on the subject TCR sequence data, a subject TCR binding pattern, and determining, based on a repository of antigen locations and the subject TCR binding pattern, a likelihood that a subject associated with the TCR sequence data has traveled to one or more locations.

The method 1400 may further comprise generating, based on the final strength of interaction data, a TCR binding pattern for a subject. The method 1400 may further comprise receiving, at a subsequent point in time, second RNA sequence data, second dextramer sequence data, and second TCR sequence data for the subject, determining, based on the second RNA sequence data, the second dextramer sequence data, and the second TCR sequence data for the subject, a second TCR binding pattern, and identifying, based on a comparison of the TCR binding pattern for the subject and the second TCR binding pattern, the subject. The method 1400 may further comprise presenting, to the predictive model, an unknown TCR sequence, wherein the predictive model and predicting, by the predictive model, a binding affinity.

### EXAMPLES

Example 1 is a method comprising: performing droplet-based single-cell RNA sequencing to generate, for each droplet, RNA sequence data, TCR sequence data, and dextramer sequence data; determining, based on a first measure of similarity, one or more TCR clusters from the TCR sequence data; determining, based on a second measure of similarity, one or more dextramer clusters from the dextramer sequence data; creating RNA data indicating a count of RNA sequences derived from one or more genes present in each cell containing droplet; creating dextramer data indicating a count of each of one or more dextramers present in each cell containing droplet; creating TCR data indicating one or more TCR sequences present in each cell containing droplet; adjusting, based on background correction derived from counts of dextramers present in non-cell containing droplets, the dextramer data; removing, based on the RNA data, data associated with a droplet comprising two or more cells and data associated with a droplet containing no TCR sequence from the dextramer data; normalizing the dextramer data; generating, based on the dextramer data, intermediate relative strength of interaction data indicating a strength of interaction for a TCR with each of one or more dextramers; removing, from the intermediate relative strength of interaction data, data that does not satisfy a threshold; aggregating, based on the one or more dextramer clusters, data of the intermediate relative strength of interaction data; removing, from the intermediate relative strength of interaction data, data that does not satisfy an interaction threshold; creating final strength of interaction data comprising data remaining in the intermediate relative strength of interaction data that indicates a strength of interaction for a TCR with each of one or more dextramer clusters; aggregating, based on the one or more TCR clusters, data of the intermediate relative strength of interaction data; removing, from the intermediate relative strength of interaction data, data that does not satisfy a clonal specificity threshold; adding, to the final strength of interaction data, data remaining in the intermediate relative strength of interaction data that indicates a strength of interaction for a TCR cluster with each of one or more dextramer clusters; and outputting the final strength of interaction data.

In Example 2, the subject matter of Example 1 includes, wherein the RNA sequence data comprises sequence data associated with one or more RNA sequences present in a droplet and gene identification data identifying a gene associated with each of the one or more RNA sequences.

In Example 3, the subject matter of Examples 1-2 includes, wherein the TCR sequence data comprises sequence data associated with one or more TCR sequences present in a droplet.

In Example 4, the subject matter of Examples 1-3 includes, wherein the dextramer sequence data comprises sequence data associated with one or more dextramer sequences present in a droplet and dextramer identification data identifying a dextramer associated with each of the one or more dextramer sequences.

In Example 5, the subject matter of Examples 1-4 includes, wherein the first measure of similarity comprises sequence similarity.

In Example 6, the subject matter of Examples 1-5 includes, wherein determining, based on a first measure of similarity, one or more TCR clusters from the TCR sequence data comprises: determining, based on aligning a plurality of TCR sequences of the TCR sequence data, a plurality of similarity scores associated with the plurality of TCR sequences; generating, based on the plurality of similarity scores, a distance matrix; and generating, based on the distance matrix, the one or more TCR clusters.

In Example 7, the subject matter of Examples 1-6 includes, wherein the second measure of similarity comprises sequence similarity.

In Example 8, the subject matter of Examples 1-7 includes, wherein determining, based on a second measure of similarity, one or more dextramer clusters from the dextramer sequence data comprises: determining, based on aligning a plurality of dextramer sequences of the dextramer sequence data, a plurality of similarity scores associated with the plurality of dextramer sequences; generating, based on the plurality of similarity scores, a distance matrix; and generating, based on the distance matrix, the one or more dextramer clusters.

In Example 9, the subject matter of Examples 1-8 includes, identifying each droplet as a cell containing droplet or a non-cell containing droplet.

In Example 10, the subject matter of Examples 1-9 includes, wherein creating RNA data indicating a count of RNA sequences derived from one or more genes present in each cell containing droplet comprises: mapping the RNA sequence data to a genome reference sequence; and determining, for each droplet, based on the mapping, the count of RNA sequences derived from one or more genes present in each droplet.

In Example 11, the subject matter of Examples 1-10 includes, wherein creating dextramer data indicating a count of each of one or more dextramers present in each cell containing droplet comprises: determining, for each droplet, based on the dextramer sequence data, the count of each of one or more dextramers present in each droplet.

In Example 12, the subject matter of Examples 1-11 includes, wherein creating TCR data indicating one or more TCR sequences present in each cell containing droplet comprises: mapping the TCR sequence data to a TCR sequence library; and determining, for each droplet, based on the mapping, the one or more TCR sequences present in each cell containing droplet.

In Example 13, the subject matter of Examples 1-12 includes, wherein adjusting, based on background correction derived from counts of dextramers present in non-cell containing droplets, the dextramer data comprises z-scaling cell containing droplets using the non-cell containing droplets.

In Example 14, the subject matter of Examples 1-13 includes, identifying a number of cells present in each cell containing droplet.

In Example 15, the subject matter of Examples 1-14 includes, identifying each droplet that contains no TCR sequence.

In Example 16, the subject matter of Examples 1-15 includes, wherein removing, based on the RNA data, data associated with a droplet comprising two or more cells and data associated with a droplet containing no TCR sequence from the dextramer data comprises deleting the data associated with a droplet comprising two or more cells and the data associated with a droplet containing no TCR sequence from a data structure containing the dextramer data.

In Example 17, the subject matter of Examples 1-16 includes, wherein normalizing the dextramer data comprises removing, from further consideration, data associated with a droplet that does not contain both an alpha chain and a beta chain; or removing, from further consideration, data associated with a droplet that does not either an alpha chain or a beta chain.

In Example 18, the subject matter of Examples 1-17 includes, wherein generating, based on the dextramer data, intermediate relative strength of interaction data indicating a strength of interaction for a TCR with each of one or more dextramers comprises a fractional signal to each dextramer in a droplet.

In Example 19, the subject matter of Examples 1-18 includes, wherein the threshold is zero, and removing, from the intermediate relative strength of interaction data, data that does not satisfy the threshold comprises deleting the data that does not satisfy the threshold from a data structure containing the dextramer data.

In Example 20, the subject matter of Examples 1-19 includes, wherein

aggregating, based on the one or more dextramer clusters, data of the intermediate relative strength of interaction data comprises collapsing one or more columns of a data structure containing the dextramer data, wherein the columns are collapsed according to dextramers contained within the same dextramer cluster.

In Example 21, the subject matter of Examples 1-20 includes, wherein the interaction threshold is configured for removal of data associated with cells having appreciable signal across a plurality of clusters of dextramers, and wherein removing, from the intermediate relative strength of interaction data, data that does not satisfy the interaction threshold comprises deleting the data that does not satisfy the interaction threshold from a data structure containing the dextramer data.

In Example 22, the subject matter of Examples 1-21 includes, wherein creating final strength of interaction data comprising data remaining in the intermediate relative strength of interaction data that indicates a strength of interaction for a TCR with each of one or more dextramer clusters comprises generating a data structure containing the final strength of interaction data.

In Example 23, the subject matter of Examples 1-22 includes, wherein aggregating, based on the one or more TCR clusters, data of the intermediate relative strength of interaction data comprises collapsing one or more rows of a data structure containing the dextramer data, wherein the rows are collapsed according to TCRs contained within the same TCR cluster.

In Example 24, the subject matter of Examples 1-23 includes, wherein the clonal specificity threshold is configured for removal of data associated with cells of a TCR cluster having weak interaction with a cluster of dextramers, and wherein removing, from the intermediate relative strength of interaction data, data that does not satisfy the clonal specificity threshold comprises deleting the data that does not satisfy the clonal specificity threshold from a data structure containing the dextramer data.

In Example 25, the subject matter of Examples 1-24 includes, wherein adding, to the final strength of interaction data, data remaining in the intermediate relative strength of interaction data that indicates a strength of interaction for a TCR cluster with each of one or more dextramer clusters comprises updating a data structure containing the final strength of interaction data with the data remaining in the intermediate relative strength of interaction data that indicates a strength of interaction for a TCR cluster with each of one or more dextramer clusters.

In Example 26, the subject matter of Examples 1-25 includes, wherein outputting the final strength of interaction data comprises displaying the final strength of interaction data on an output device.

In Example 27, the subject matter of Examples 1-26 includes, training a predictive model based on the final strength of interaction data.

In Example 28, the subject matter of Example 27 includes, predicting a binding status of a newly presented TCR sequence according to the trained predictive model.

In Example 29, the subject matter of Examples 27-28 includes, presenting, to the predictive model, subject TCR sequence data; determining, by the predictive model, based on the subject TCR sequence data, a subject TCR binding pattern; and determining, based on a repository of antigen locations and the subject TCR binding pattern, a likelihood that a subject associated with the TCR sequence data has traveled to one or more locations.

In Example 30, the subject matter of Examples 1-29 includes, generating, based on the final strength of interaction data, a TCR binding pattern for a subject.

In Example 31, the subject matter of Example 30 includes, receiving, at a subsequent point in time, second RNA sequence data, second dextramer sequence data, and second TCR sequence data for the subject; determining, based on the second RNA sequence data, the second dextramer sequence data, and the second TCR sequence data for the subject, a second TCR binding pattern; and identifying, based on a comparison of the TCR binding pattern for the subject and the second TCR binding pattern, the subject.

In Example 32, the subject matter of Examples 27-31 includes, presenting, to the predictive model, an unknown TCR sequence, wherein the predictive model; and predicting, by the predictive model, a binding affinity.

Example 33 is at least one machine-readable medium including instructions that, when executed by processing circuitry, cause the processing circuitry to perform operations to implement of any of Examples 1-32.

Example 34 is an apparatus comprising means to implement of any of Examples 1-32.

Example 35 is a system to implement of any of Examples 1-32.

Example 36 is a method to implement of any of Examples 1-32.

Example 37 is a method comprising: determining, for each of a plurality of droplets ribonucleic acid (RNA) sequence data, T-cell receptor (TCR) sequence data, and dextramer sequence data; determining, based on the dextramer sequence data and droplets of the plurality of droplets comprising a single cell and at least one TCR sequence, dextramer data indicating a count of each of the one or more dextramers present in each cell-containing droplet of the plurality of droplets; generating, based on the dextramer data, intermediate relative strength of interaction data indicating a clonal specificity threshold and indicating a strength of interaction for a TCR satisfying a threshold with each of the one or more dextramers; aggregating, based on one or more dextramer clusters of the one or more dextramer having a measure of similarity determined based on the dextramer sequence data, data of the intermediate relative strength of interaction data into final strength of interaction data; and outputting the final strength of interaction data.

In Example 38, the subject matter of Example 37 includes, wherein the RNA sequence data comprises sequence data associated with one or more RNA sequences present in a droplet of the plurality of droplets and gene identification data identifying a gene associated with each of the one or more RNA sequences.

In Example 39, the subject matter of Examples 37-38 includes, wherein the TCR sequence data comprises sequence data associated with one or more TCR sequences present in a droplet of the plurality of droplets.

In Example 40, the subject matter of Examples 37-39 includes, wherein the dextramer sequence data comprises sequence data associated with one or more dextramer sequences present in a droplet of the plurality of droplets and dextramer identification data identifying a dextramer associated with each of the one or more dextramer sequences.

In Example 41, the subject matter of Examples 37-40 includes, determining, based on the measure of similarity, the one or more dextramer clusters from the dextramer sequence data.

In Example 42, the subject matter of Examples 37-41 includes, determining, based on a second measure of similarity, one or more TCR clusters from the TCR sequence data; and aggregating, based on the one or more TCR clusters, the data of the intermediate relative strength of interaction data for inclusion in the final strength of interaction data.

In Example 43, the subject matter of Example 42 includes, wherein determining, based on the measure of similarity, one or more TCR clusters from the TCR sequence data comprises: determining, based on aligning a plurality of TCR sequences of the TCR sequence data, a plurality of similarity scores associated with the plurality of TCR sequences; generating, based on the plurality of similarity scores, a distance matrix; and generating, based on the distance matrix, the one or more TCR clusters.

Example 44 is at least one machine-readable medium including instructions that, when executed by processing circuitry, cause the processing circuitry to perform operations to implement of any of Examples 37-43.

Example 45 is an apparatus comprising means to implement of any of Examples 37-43.

Example 46 is a method comprising: determining, for each of a plurality of droplets ribonucleic acid (RNA) sequence data, T-cell receptor (TCR) sequence data, and dextramer sequence data; determining, based on the dextramer sequence data and droplets of the plurality of droplets comprising a single cell and at least one TCR sequence, dextramer data indicating a count of each of the one or more dextramers present in each cell-containing droplet of the plurality of droplets; generating, based on the dextramer data, intermediate relative strength of interaction data indicating a clonal specificity threshold and indicating a strength of interaction for a TCR satisfying a threshold with each of the one or more dextramers; aggregating, based on one or more TCR clusters having a measure of similarity determined based on the TCR sequence data, data of the intermediate relative strength of interaction data into final strength of interaction data; and output the final strength of interaction data.

In Example 47, the subject matter of Example 46 includes, wherein the RNA sequence data comprises sequence data associated with one or more RNA sequences present in a droplet of the plurality of droplets and gene identification data identifying a gene associated with each of the one or more RNA sequences.

In Example 48, the subject matter of Examples 46-47 includes, wherein the TCR sequence data comprises sequence data associated with one or more TCR sequences present in a droplet of the plurality of droplets.

In Example 49, the subject matter of Examples 46-48 includes, wherein the dextramer sequence data comprises sequence data associated with one or more dextramer sequences present in a droplet of the plurality of droplets and dextramer identification data identifying a dextramer associated with each of the one or more dextramer sequences.

In Example 50, the subject matter of Examples 46-49 includes, determining, based on the measure of similarity, the one or more TCR clusters from the TCR sequence data.

In Example 51, the subject matter of Examples 46-50 includes, determining, based on a second measure of similarity, one or more dextramer clusters from the dextraner sequence data; and aggregating, based on the one or more dextramer clusters, the data of the intermediate relative strength of interaction data for inclusion in the final strength of interaction data.

In Example 52, the subject matter of Example 51 includes, wherein determining, based on the second measure of similarity, the one or more dextramer clusters from the dextramer sequence data comprises: determining, based on aligning a plurality of dextramer sequences of the dextramer sequence data, a plurality of similarity scores associated with the plurality of dextramer sequences; generating, based on the plurality of similarity scores, a distance matrix; and generating, based on the distance matrix, the one or more dextramer clusters.

Example 53 is at least one machine-readable medium including instructions that, when executed by processing circuitry, cause the processing circuitry to perform operations to implement of any of Examples 46-52.

Example 54 is an apparatus comprising means to implement of any of Examples 46-52.

Example 55 is a system to implement of any of Examples 46-52.

Example 56 is a method to implement of any of Examples 46-52.

Those skilled in the art will recognize, or be able to ascertain using no more than routine experimentation, many equivalents to the specific embodiments of the method and compositions described herein. Such equivalents are intended to be encompassed by the following claims.

We claim:

1. A method comprising:
determining, for each of a plurality of droplets ribonucleic acid (RNA) sequence data, T-cell receptor (TCR) sequence data, and dextramer sequence data;
determining, based on the dextramer sequence data and droplets of the plurality of droplets comprising a single cell and at least one TCR sequence, dextramer data indicating a count of each of the one or more dextramers present in each cell-containing droplet of the plurality of droplets;
generating, based on the dextramer data, intermediate relative strength of interaction data indicating a clonal specificity threshold and indicating a strength of interaction for a TCR satisfying a threshold with each of the one or more dextramers;
aggregating, based on one or more dextramer clusters of the one or more dextramer having a measure of similarity determined based on the dextramer sequence data, data of the intermediate relative strength of interaction data into final strength of interaction data; and
outputting the final strength of interaction data.

2. The method of claim 1, wherein the RNA sequence data comprises sequence data associated with one or more RNA sequences present in a droplet of the plurality of droplets and gene identification data identifying a gene associated with each of the one or more RNA sequences.

3. The method of claim 1, wherein the TCR sequence data comprises sequence data associated with one or more TCR sequences present in a droplet of the plurality of droplets.

4. The method of claim 1, wherein the dextramer sequence data comprises sequence data associated with one or more dextramer sequences present in a droplet of the plurality of droplets and dextramer identification data identifying a dextramer associated with each of the one or more dextramer sequences.

5. The method of claim 1, further comprising determining, based on the measure of similarity, the one or more dextramer clusters from the dextramer sequence data.

6. The method of claim 1, further comprising:
determining, based on a second measure of similarity, one or more TCR clusters from the TCR sequence data; and
aggregating, based on the one or more TCR clusters, the data of the intermediate relative strength of interaction data for inclusion in the final strength of interaction data.

7. The method of claim 6, wherein determining, based on the second measure of similarity, the one or more TCR clusters from the TCR sequence data comprises:
determining, based on aligning a plurality of TCR sequences of the TCR sequence data, a plurality of similarity scores associated with the plurality of TCR sequences;
generating, based on the plurality of similarity scores, a distance matrix; and
generating, based on the distance matrix, the one or more TCR clusters.

8. A system comprising:
a first computing device configured to:
determine, for each of a plurality of droplets ribonucleic acid (RNA) sequence data, T-cell receptor (TCR) sequence data, and dextramer sequence data;
determine, based on the dextramer sequence data and droplets of the plurality of droplets comprising a single cell and at least one TCR sequence, dextramer data indicating a count of each of the one or more dextramers present in each cell-containing droplet of the plurality of droplets;
generate, based on the dextramer data, intermediate relative strength of interaction data indicating a clonal specificity threshold and indicating a strength of interaction for a TCR satisfying a threshold with each of the one or more dextramers;
aggregate, based on one or more TCR clusters having a measure of similarity determined based on the TCR sequence data, data of the intermediate relative strength of interaction data into final strength of interaction data; and
output the final strength of interaction data; and
a second computing device configured to receive the final strength of interaction data.

9. The system of claim 8, wherein the RNA sequence data comprises sequence data associated with one or more RNA sequences present in a droplet of the plurality of droplets and gene identification data identifying a gene associated with each of the one or more RNA sequences.

10. The system of claim 8, wherein the TCR sequence data comprises sequence data associated with one or more TCR sequences present in a droplet of the plurality of droplets.

11. The system of claim 8, wherein the dextramer sequence data comprises sequence data associated with one or more dextramer sequences present in a droplet of the plurality of droplets and dextramer identification data identifying a dextramer associated with each of the one or more dextramer sequences.

12. The system of claim 8, further comprising determining, based on the measure of similarity, the one or more TCR clusters from the TCR sequence data.

13. The system of claim 8, wherein the first computing device configured to:
determine, based on a second measure of similarity, one or more dextramer clusters from the dextraner sequence data; and
aggregate, based on the one or more dextramer clusters, the data of the intermediate relative strength of interaction data for inclusion in the final strength of interaction data.

14. The system of claim 13, wherein the first computing device configured to determine, based on the second measure of similarity, the one or more dextramer clusters from the dextramer sequence data comprises the first computing device configured to:
determine, based on aligning a plurality of dextramer sequences of the dextramer sequence data, a plurality of similarity scores associated with the plurality of dextramer sequences;
generate, based on the plurality of similarity scores, a distance matrix; and
generate, based on the distance matrix, the one or more dextramer clusters.

15. One or more non-transitory computer-readable media storing processor-executable instructions that, when executed by at least one processor, cause the at least one processor to:
determine, for each of a plurality of droplets ribonucleic acid (RNA) sequence data, T-cell receptor (TCR) sequence data, and dextramer sequence data;
determine, based on the dextramer sequence data and droplets of the plurality of droplets comprising a single cell and at least one TCR sequence, dextramer data indicating a count of each of the one or more dextramers present in each cell-containing droplet of the plurality of droplets;
generate, based on the dextramer data, intermediate relative strength of interaction data indicating a clonal specificity threshold and indicating a strength of interaction for a TCR satisfying a threshold with each of the one or more dextramers;
aggregate, based on one or more dextramer clusters of the one or more dextramer having a measure of similarity determined based on the dextramer sequence data, data of the intermediate relative strength of interaction data into final strength of interaction data; and
output the final strength of interaction data.

16. The one or more non-transitory computer-readable media of claim 15, wherein the RNA sequence data comprises sequence data associated with one or more RNA sequences present in a droplet of the plurality of droplets and gene identification data identifying a gene associated with each of the one or more RNA sequences.

17. The one or more non-transitory computer-readable media of claim 15, wherein the TCR sequence data comprises sequence data associated with one or more TCR sequences present in a droplet of the plurality of droplets.

18. The one or more non-transitory computer-readable media of claim 15, wherein the dextramer sequence data comprises sequence data associated with one or more dextramer sequences present in a droplet of the plurality of droplets and dextramer identification data identifying a dextramer associated with each of the one or more dextramer sequences.

19. The one or more non-transitory computer-readable media of claim 15, wherein the processor-executable instructions further cause the at least one processor to:
determine, based on a second measure of similarity, one or more TCR clusters from the TCR sequence data; and
aggregate, based on the one or more TCR clusters, the data of the intermediate relative strength of interaction data for inclusion in the final strength of interaction data.

20. The one or more non-transitory computer-readable media of claim 19, wherein the processor-executable instructions that cause the at least one processor to determine, based on the second measure of similarity, the one or more TCR clusters from the TCR sequence data further cause the at least one processor to:
determine, based on aligning a plurality of TCR sequences of the TCR sequence data,
a plurality of similarity scores associated with the plurality of TCR sequences;
generate, based on the plurality of similarity scores, a distance matrix; and
generate, based on the distance matrix, the one or more TCR clusters.
